(19) United States
(12) Patent Application Publication
(10) Pub. No.: US 2014/0216158 A1
(43) Pub. Date: Aug. 7, 2014

(54) AIR COUPLED ULTRASONIC CONTACTLESS METHOD FOR NON-DESTRUCTIVE DETERMINATION OF DEFECTS IN LAMINATED STRUCTURES

(71) Applicants: Sergio José Sanabria Martin, Zurich (CH); Jürg Neuenschwander, Grut (CH); Urs Sennhauser, Meilen (CH)

(72) Inventors: Sergio José Sanabria Martin, Zurich (CH); Jürg Neuenschwander, Grut (CH); Urs Sennhauser, Meilen (CH)

(21) Appl. No.: 14/238,895

(22) PCT Filed: Aug. 9, 2012
(86) PCT No.: PCT/EP2012/065593

§ 371 Date: Mar. 31, 2014

(30) Foreign Application Priority Data
Aug. 17, 2011 (CH) 01354/11

(51) Int. Cl. G01N 33/46; G01N 29/06

(52) U.S. Cl. CPC G01N 33/46; G01N 29/06
USPC 73/588

(57) ABSTRACT

There is an air coupled ultrasonic contactless method and an installation for non-destructive determination of defects in laminated structures with a width (W) and a multiplicity of n lamellas with intermediate N−1 bonding plants (B), whereas at least one transmitter (T) in a fixed transmitter distance (WTS) radiates ultrasound beams at multiple positions and at least one receiver (R) in a sensor distance (WSR) is receiving re-radiated ultrasound beams at multiple positions relative to the laminated structure (S). The method images the position and geometry of for example lamination defects and allows for inspection of laminated structure (S) of arbitrary height (H) and length (L), and an individual assessment of specific bonding planes (e.g. B1, B2, B3), as well in situations with constrained access to the faces of the sample parallel to the bonding planes.

### CROSS-REFERENCE TO RELATED APPLICATIONS

This application claims priority to International Application Serial No. PCT/EP2012/065593, filed Aug. 9, 2012, which claims priority to Swiss Patent Application No. CH 01354/11 filed Aug. 17, 2011. International Application Serial No. PCT/EP2012/065593 is hereby incorporated herein for all purposes by this reference.

### FIELD OF THE INVENTION

The present invention describes an air coupled ultrasonic contactiess method for non-destructive determination of defects in laminated structures with a width and a multiplicity of n lamellas with intermediate n−1 bonding planes, whereas at least one transmitter in a fixed transmitter distance radiates ultrasound incident sound fields at multiple positions and at least one receiver in a sensor distance is receiving re-radiated ultrasound fields at multiple positions relative to the laminated structure, the use of the method and an installation for carry out the method.

### BACKGROUND

There is a commercial need for laminated structures for example for glued timber structures existing as a building material. Wood is a renewable building material with a high ecological and economic value. The conventional forms of sawn wood in construction have been replaced by laminated structures comprising wood or wood composite materials for example glued timber products which allow for versatile structural members with increased load-carrying capacity.

A common, but not the only, manufacture process consists of drying, planing, end-jointing, gluing and stacking selected timber lamellas (each typically 30 to 40 mm thick, up to 200 mm wide and 1000 mm long) into glued laminated timber glulam beams, each typically one lamella wide, up to 2000 mm high in the stacking direction and large lengths (>50 m) in a straight or curved form.

In order to maximize the life cycle of the constructions and to prevent security hazards, it is necessary that the integrity and load bearing capacity of the glue lines are tested. Possible testing environments are a global quality assessment implemented in glued timber production lines, or in situ testing at existing constructions by means of periodical inspections aimed at early identification of cracking and delamination, which are caused, among others, by climate induced internal stresses and long-lasting loads.

Currently standardized testing methods rely on destructive mechanical tests in random specimens, visual inspection, and for this application relevant non-destructive testing methods using ultrasound.

Ultrasonic diagnostics are well-suited for the assessment of laminated structures as glued timber due to their high sensitivity to delaminated interfaces. Traditional inspections are performed with transducers pressed onto the sample (dry or with a coupling gel) in a contact ultrasound measurement. The main disadvantages are a low reproducibility, due to varying coupling pressure and possible damage of the inspected surfaces. Moreover, ultrasound imaging of defect positions and geometries in contact ultrasound mode is highly time-consuming, since continuous scanning of ultrasonic transducers is generally not possible.

Since a few years contactless air coupled ultrasonic non-destructive determination of defects in laminated structures is used, which overcomes the described difficulties. The main challenge of contactless ultrasonics is that most of transmitted energy is specularly reflected at interfaces air-wood, so that only very small ultrasound signals are coupled through the sample. Special developed transducers, receivers and dedicated low-noise electronics were implemented in recent years which are capable to conduct measurements in spite of the small percentage of ultrasound pressure in air (<0.5% for wood) which is coupled into the inspected laminated structures and, in the case of timber, the high attenuation of ultrasound in the inspected laminated structures (23 dB cm⁻¹ MHz⁻¹ for wood).

As shown in FIG. 1 (prior art) transducers are held at some distance separated with an air gap A from the glued multilayer timber sample S allowing for non-invasive reproducible scanning. Currently available systems are intended for the inspection of plate specimens of wood and wood based composites (e.g. fiberboard, particleboard, plywood) up to a few centimeter height H.

A transmitter T and a receiver transducer R are located on both sides of the sample facing the long side faces FPi of the plate. The plate is insonified by ultrasound beams being essentially perpendicular to one long side face FP1 of the plate, to the laminated bonding planes B1, B2 and possible defect areas D due to imperfect lamination as shown in FIGS. 1a and 1b. A reduction of transmitted ultrasound energy between T and R is typically observed in the presence of defects and can be used for determination of defects.

As disclosed in EP1324032 the air coupled ultrasound beam used for determination of defects in bonding of plane sheet elements is inclined relative to the normal of the long side face of the board to be tested. Using the method of EP1324032 for testing laminated structures with a multiplicity of lamellas does not lead to desired results for determination of the location of bonding defects.

Also a single-sided inspection configuration depicted in prior art as known from U.S. Pat. No. 5,824,908 for instance, couples guided waves in a few mm thick sheet specimens (e.g. paper, thin veneer) for material properties characterization. The inclined insonification relative to the normal of the long side face of a monolayer board can be determined by at least one receiver. To be able to characterize defects in the board the setup has to be operated near the resonant frequency. This setup is either not feasible or provides very limited information when applied to glued timber members of large cross-section (H>30 cm) as, for instance, glued laminated timber beams.

In Sanabria et al. “Air-coupled ultrasound inspection of glued laminated timer”, Holzforschung, Vol. 65, pp. 377-387, 2011, measurement setups for non-destructive evaluation of glulam beams with different numbers of lamellas are shown. The insonification relative to the normal of the long side face of a glulam beam with one transmitter and one receiver is disclosed. The transmitter and the receiver are scanned in normal transmission mode in X (beam length) and Y (beam width) directions parallel to the bonding planes of the timber lamellas. With a special developed low-noise electronics, tuned transmitter and sensitive receiver assessment of glulam is possible.

In many real situations, for example glued timber bending members used in roofing applications, access to the surfaces of the sample parallel to the bonding planes is constrained by a limiting layer, therefore the known setups are not feasible for in situ application.

For a laminated structure with multiplicity of lamellas, the known methods can only determine whether a stack of bonding planes is defective or defect-free. The known methods provide no or very limited information about which specific lamella or bonding plane (e.g. B1, B2) of the stack is defective. This information is critical to quantify the structural relevance of the defect and to decide whether either no action, a specific reinforcement of the structure or a full replacement of the faulty member is required. For example, in a typical glulam beam highest bending stresses are located in the outmost laminations. Therefore, the known setups do not lead to desired results for determination of the location of bonding defects.

The complexity of acquisition and analysis of ultrasound data increases exponentially with the thickness of the laminate. Adding material attenuation to the air-coupling losses, the pressure level transmitted through 300 mm glued timber made is reduced more than 500'000 times with respect to direct air transmission, so that the transmitted signal level is typically under the noise floor of state-of-the-art electronics. Moreover, spurious waves (e.g. diffracted waves through air and transducer holders) become larger than the signal transmitted through wood. Besides, the wave path in glued timber is subjected to position, amplitude and phase uncertainties which are accumulative with the number of insonified timber lamellas.

Finally, scattering and mode conversion phenomena occur in each lamination, meaning that the number of propagation paths 3^{NB} increases exponentially with the number of lamination interfaces N_{B} crossed by the ultrasound beam, which further increases the uncertainty of the assessment as known from Sanabria et al. The limit of measurability is reached and can only be expanded with much more further technical developments in transducer technology and the low-noise electronics in order to analyze thicker laminated structures comprising more timber lamellas.

### DESCRIPTION OF EXEMPLARY EMBODIMENTS THE INVENTION

The object of the present invention is to establish a method which overcomes the limitations described above, suitable for flaw monitoring of defects in laminated structures, in particular timber defects in glued timber structural members of large cross-section comprising a multiplicity of lamellas, at the production process or in situ in implemented condition. This determination of lamination defects is using advanced air-coupled ultrasound imaging.

Another object of the present invention is to provide an installation for the determination of lamination flaws of glued laminated timber beams.

More specifically, the objective of the invention is to allow for assessment of glued timber structural members of arbitrary height and length, and an individual assessment of defects of specific bonding planes, as well as in situations with constrained access to the long side face of the sample parallel to the bonding planes by a limiting layer.

The objective is achieved by a method for non-destructive determination of defects of glued timber members of large cross-section, for example, glued laminated timber beams, which images the position and geometry of timber defects such as, in particular, lamination flaws, for individual bonding planes, as well in situations with limited access to the long side face of the sample parallel to the bonding planes, and which is summarized by the following steps:
a) Generation of an ultrasound beam in air and coupling into a glued timber member through at least one of the two accessible lateral faces of the sample which are essentially perpendicular to the inspected bonding planes, the ultrasound beam being tilted with a small inclination (typically 0 to 20° for wood) from the normal of the insonified surface, such that a refracted beam at a desired inclination is generated within the structural member.
b) Adjustment of the ultrasound beam position and orientation along predetermined paths in order to scan the entire glued timber structural member along the lateral face.
c) Acquisition of specific wave propagation paths from the sound field transmitted or reflected through the glued timber structural member and radiated into air for each ultrasound beam position and orientation.
d) Extraction of wave signatures from the recorded ultrasound dataset describing the interaction of the ultrasound beam with defect areas, typically characterized by transmission of the ultrasound beam through defect-free regions and reflection or scattering at defect regions.
e) Determination of maps for each individual bonding plane and adjacent lamellas, in which defects are imaged, based on predetermined relations between parameters of incident ultrasound beam, ultrasound wave signatures recorded for a specific portion of the height of the sample and type, position and geometry of defects.

The objective is further achieved by an installation for non-destructive assessment of glued timber members of large cross-section as described above, which uses advanced air-coupled ultrasound technologies comprising:
a. An air-coupled ultrasound beam generator consisting, for instance, of a computerized arbitrary waveform generator connected to an amplifying unit for generating a high-voltage, typically pulsed, electrical signal, which is then transformed into a space-limited air pressure field in ultrasound frequencies by an excitation unit consisting of a defined number of ultrasonic transmitter transducers separated with a variable air gap from the insonified surface of the inspected glued timber sample.
b. A device, e.g. mechanical or electronic scanners together with transducer positioning fixtures, which changes the position and orientation of the ultrasound beam along defined trajectories over a defined inspection region and selects specific wave propagation paths through the glued timber sample by adjusting parameters, e.g. position, orientation and phasing of transmitter and receiver ultrasonic transducers and associated excited and received signals down to a desired level of accuracy.
c. A non-contact receiver unit composed, for instance, of a sensor unit consisting of ultrasonic receiver transducers separated with an air gap from the glued timber sample which transform the reflected/transmitted air pressure field into electrical signals, low-noise amplification and filtering electronics for conditioning transduced ultrasonic signals, and analogue to digital conversion for time/space digitization of datasets consisting of ultrasound waveforms for each scanned position.
d. Specific signal processing algorithms implemented on a microprocessor for noise and spurious signal reduction together with ultrasonic wave signature extraction.
e. Specific direct and inverse ultrasound wave propagation models for derivation of optimum experimental parameters such as transducer positions and orientations, correlation of acquired wave signatures with ultrasound propagation paths within glued timber sample, and inversion of sound field at defect areas, from which type, position and geometry of defects are derived.

The coupling of the ultrasound beam and the acquisition of the re-radiated ultrasound field can be performed through one or different lateral faces of the specimen.

An ultrasound beam can be coupled and read at discrete positions and orientations and the ultrasound energy transmitted through specific wave propagation paths, pre-calculated with a direct wave propagation model, is associated to the presence or absence of defects of certain type and geometry along the wave propagation paths.

For an arbitrary incident ultrasound beam/sound field coupled into the sample, which propagates through the sample and interacts with defects, it is possible to determine the total sound field re-radiated into air. The received data can then be used as input to a computational inverse wave propagation model from which the sound field at defect areas and inner wave propagation paths within the sample are inverted and defect positions, types and geometries are determined.

A space diversity setup is usable that simultaneously provides multiple independent observations of a single timber defect area, each observation being associated to a differentiated wave propagation path. The combination of multiple observations is used to improve defect characterization and differentiation with respect to the natural variability of the sample, and provides a verification means of the assessment. The percentage of agreeing observations gives a measurement of the robustness of the assessment,

With the disclosed method periodic monitoring of the sample under test can be performed and differences between successive measurements are identified and associated to the development of defects.

### BRIEF DESCRIPTION OF THE DRAWINGS

Preferred embodiments of the subject matter of the invention is described below in conjunction with the attached drawings, whereas the laminated structures are timber structures.

FIGS. 1 a) and b) are showing schematic sectional views of known air-coupled ultrasound assessments of timber structures comprising at least one lamella according to prior art.

FIG. 2 shows a schematic representation of a specific installation for non-destructive testing of lamination defects in glued laminated timber beams of large cross section using advanced air-coupled ultrasound imaging according to the method presented by the invention, optionally a wave propagation model can be used.

FIGS. 3 a) and b) are showing double-sided inspection setups, in which two opposing lateral faces of the tested sample are used for ultrasound beam coupling and sound field reception, respectively, with a) negative detection and b) positive detection.

FIGS. 4 a) to c) are showing single-sided inspection setups, in which the same lateral face of the sample is used for both ultrasound beam coupling and sound field reception.

FIG. 5 a) is showing schematic representation of a specific space diversity setup in which multiple inspection configurations of FIG. 3 are combined with a logic table (FIG. 5b) into a single assessment

FIG. 6 a) to f) are showing specific realization of excitation and sensor units with single and multiple transducer elements.

FIG. 7 shows a specific implementation of transducer positioning fixture unit a) in different side views, and b) side views of two transducer positioning fixture units connected to a scanning system unit and c) a perspective view of an installation for performing the method.

FIGS. 8 a) and b) are showing schematic representation of specific direct and inverse ultrasound wave propagation models, respectively, for timber defect localization and characterization.

FIG. 9 a1) shows a schematic representation of a double-sided inspection setup in negative detection mode according to FIG. 3a, a2) shows a measured ultrasound image generated from a multiplicity of time waveforms recorded for each position of the image, from which two specific time waveforms a3) for a4) at defect and defect-free positions, respectively, are shown, in comparison with a calculated expected ultrasound image a5) while Figure b1) to b5) are showing drawings on the basis of FIGS. 9 a1) to a5) while a double-sided inspection setup in negative detection mode with a different angle of insonification is used.

FIGS. 10 a1) to a5) are showing drawings on the basis of FIGS. 9 a1) to a5) while positive detection mode is used according to FIG. 3b.

FIGS. 11 a1) shows a schematic representation of a double-sided inspection setup for characterization of total re-radiated sound field according to FIG. 6c, where a directive transducer is used as receiver, a2) shows a measured ultrasound image generated from a multiplicity of time waveforms recorded for each position of the image, from which two specific time waveforms a3) and a4) at to specific positions are shown, while FIG. 1 b1) to b4) are showing drawings on the basis of FIGS. 11a1) to a4) while a ominidirectional transducer is used as receiver.

FIGS. 12 a) to f) are showing ultrasound beam paths in schematic representation calculated by theoretic models.

### DESCRIPTION OF EXEMPLARY EMBODIMENTS OF THE INVENTION

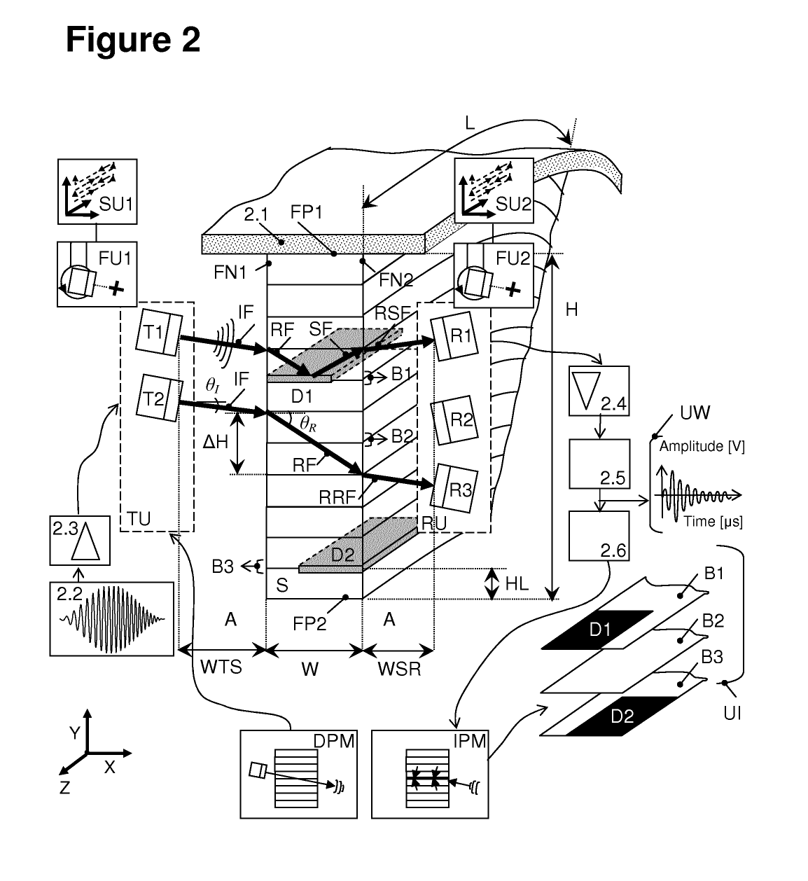

The present ultrasound inspection method is explained according to the example of FIG. 2. In this application the determination of defects D in laminated structures S is explained for example by description of determination of timber defects D, D1, D2, . . . Di in glued laminated timber structures S. The laminated structure S features a height H, a length L and a width W. The laminated structure S is built by a multiplicity of timber lamellas which are interconnected (glued) in a multiplicity of bonding planes B, B1, B2 and B3, whereas each lamella is possessing a defined lamella height HL or mean timber lamella thickness HL.

The interaction between ultrasound beam and timber defect areas generates specific physical phenomena, namely ultrasound wave signatures, which can be identified in the recorded ultrasound datasets and associated to the presence or absence of timber defects D. A typical ultrasound wave signature is a complete or partial blocking of the sound field transmitted across the defect. Another typical ultrasound signature is the scattering or reflection of a significant amount of ultrasonic energy at the defect interfaces in a diverging direction from the transmission path associated that is associated with defect-free material. Additional ultrasound signatures are, among others, an increase of time of flight for an ultrasonic wave diffracted around a timber defect and an enhancement or cancellation of transmission at specific frequencies of the Fourier spectrum of the recorded ultrasound signals, due to interference resonance or non-linear phenomena in lamination discontinuities.

Defects D, D1, D2 are defined as starved joints, delamination, cracking, splits, voids, decay, or any undesired anomaly in the laminated structure S capable of modifying an incident sound field IF of a transmitted ultrasound beam in such a way that the ultrasound signals can be differentiated from a situation in which no defect is present. Defect areas D are defined as regions of the sample S, typically bonding planes B1, B2 and B3 or adjacent timber lamellas, where such defects D are present or suspected and wished to be assessed.

A schematic installation for the assessment of lamination flaws is shown in FIG. 2 in which an ultrasound excitation unit TU, which is driven by defined electric signals, generates an incident sound field IF which is then coupled into a laminated structure S embodied as a glued timber sample of large cross-section. The ultrasound excitation unit TU comprises at least one pivotable transmitter T, T1, T2. In this application transmitter T and later mentioned receiver are defined synonymously as transducer and are communicating with the later described electronic components.

A waveform generator 2.2 generates computerized arbitrary predefined waveform shapes which are then amplified and filtered by high power transmitter electronics 2.3 and fed to the ultrasound excitation unit TU to be insonified by the at least one transmitter T.

The at least one transmitter T is insonifying the incident sound field IF under an inclined insonification angle or inclination angle θ_{I} on a lateral face FN1 intruding into the laminated structure S at least approximately parallel to the bonding planes B. The insonification angle θ_{I} is included between the normal of the lateral face FN1 and the normal of the active face of the transmitter T.

Due to a transmitter distance WTS between the active surface of the transmitter T1, T2 and the insonified lateral face FN1, the ultrasound beam is travelling through a gap of gas medium A before entering the laminated structure S. The coupling of ultrasound energy into the sample S is achieved with the variable separation WTS between the active surface of the transmitter T, T1, T2 and the insonified lateral face FN1.

The ultrasound excitation unit TU is connected to a transducer positioning fixture unit FU1, which is attached to a scanning system unit SU1 and allows the at least one transmitter T1 to scan from a fixed a transmitter distance WTS that is at least parallel to the lateral face FN1.

The incident sound field IF is coupled through the lateral face FN1 leading to a refracted sound field RF inside the sample S. If the refracted sound field RF is hitting, then a defect D a scattered/reflected sound field SF occurs which travels to the re-radiated lateral face FN2 and couples out of the sample as a re-radiated scattered sound field RSF. If the refracted sound field RF does not hit a defect D, then an unscattered re-radiated refracted sound field RRF is coupled out of the sample. After passage of the ultrasound beam through the laminated structure S or reflection of the ultrasound beam in the laminated structure S, the ultrasound beam is leaving the sample S through a re-radiated lateral face FN2. This re-radiated lateral face FN2 could be identical with the lateral face FN1 in case of reflection of the ultrasound beam.

The re-radiated scattered sound field RSF and/or the re-radiated refracted sound field RRF can be read out with an ultrasound sensor unit RU comprising at least one receiver R, R1, R2, R3 disposed at a defined receiver distance WSR from the re-radiated lateral face FN2. The receiver distance WSR is defined by the distance between the active surface of the receiver R, R1, R2, R3 and the re-radiated lateral face FN2. The ultrasound sensor unit RU is connected to another transducer positioning fixture unit FU2, which can be scanned by another scanning system unit SU2.

The transmitter T and the receiver R can be positioned either on opposing sides of the sample as shown in FIG. 2 or on the same side as shown later. The gaps between transmitter T and lateral face FN1 and between receiver R and re-radiated lateral face FN2 are filled by an arbitrary gaseous medium, typically air. The lateral face FN1 and the re-radiated lateral face FN2 are defined as interfaces wood/air where acoustic energy is coupled to or radiated from, respectively. Some flatness and smoothness is required in these lateral faces FN1, FN2 for sufficient ultrasound energy coupling. At least one of the lateral faces FN1 and the re-radiated lateral face FN2 is essentially perpendicular to the bonding planes B, B1, B2, B3, . . . Bi respectively defect areas D inspected.

Due to pivoting of the transmitter T the coupled incident sound field IF is tilted with a small inclination angle θ_{I}, typically between 0° and 20° for wood, from the normal of the insonified lateral surface FN1. θ_{I} is chosen such that a refracted beam RF at a desired inclination θ_{R} is generated inside the sample. The ultrasound propagation then occurs through the width of the sample W and a limited portion ΔH of the height H of the sample S, which allows for an individual assessment of timber defect areas D, D1, D2 associated to specific bonding planes B, B1, B2, B3 in samples S of arbitrary height H and length L. ΔH is typically between zero and the width W of the sample S.

ΔH is typically chosen as a multiple of the average thickness of timber lamellas HL.

Since the parallel longitudinal faces FP1 and FP2 of the sample S parallel to the bonding planes B are not used for ultrasound insonification or read-out, the system can be applied in situations with constrained access to the longitudinal faces FP1, FP2 by a limiting layer 2.1, which are common for instance in roofing applications.

A minimum of two ultrasound transducers, one transmitter T and one receiver R, are required in order to implement the ultrasound excitation unit TU and the ultrasound sensor unit RU, respectively.

With the at least one receiver R of the ultrasound sensor unit RU ultrasound signals are acquired at discrete positions in an adjustable fixed sensor distance WSR from the re-radiating lateral face FN2. The measured signals are digitized for further data analysis, from which ultrasound images UI of timber defects D1, D2 in the bonding planes B1, B2 and B3 are computed.

Beside using an array of receivers R as will be described later, scanning of the at least one receiver R along a path in a plane parallel to the re-radiated lateral surface FN2 at an adjusted sensor distance WSR is preferred.

A scanning system unit SU, SU1, SU2, . . . SUi is used to continuously adjust the ultrasound beam position and orientation from the at least one transmitter T along predefined trajectories over defined regions in a plane parallel to the lateral face FN1 and/or re-radiating face FN2 of the inspected glued timber sample S and to capture at specific positions a portion of the sound field re-radiated into air on re-radiated air paths RRF, RSF after interaction with defects D. The scanning system unit SU is controlling the ultrasound excitation unit TU and the ultrasound sensor unit RU.

The scanning can be implemented with mechanical (multiple axes scanner or robotic arms) or electronic (array phasing) technologies, which adjust specific parameters of transmitter and receiver ultrasound transducers T, R and associated electronics, such as, for instance, transducer position and orientation or phasing of excited and recorded signals.

The sound field transmitted/reflected through timber defect areas D and re-radiated to the re-radiated air path RRF, RSF is transformed by the non-contact ultrasound sensor unit RU, for example ultrasound transducers, into low-level electrical signals (typically of the order of magnitude of μV) which are then amplified, filtered and conditioned with low-noise receiver electronics 2.4.

Battery powered amplifiers are generally preferred in the first amplification step and switched-mode power supplies are generally avoided in order to minimize mains electrical noise. Coaxial cabling, well-defined grounding and electrical isolation are carefully implemented for transducers and electrical components in order to obtain sufficient electromagnetic shielding and minimize noise and spurious signals, e.g. induced by ground loops.

The conditioned electrical signals are sampled, with an analogue to digital converter 2.5 and stored in a digital support at all scanned positions as ultrasound datasets for further data analysis. The measured ultrasound signals for each scanned position are forming ultrasound waveforms UW shown for example as amplitude vs. time diagrams.

Signal processing algorithms are implemented on a microprocessor 2.6 for noise and spurious signal reduction together with ultrasonic wave signature extraction.

The data processing strategies for the recorded waveforms involve some or all of the following: amplitude or phase tracking within a time window T_{R}, linear filtering, spatial processing, Fourier spectral analysis, wavelet decomposition, waveform correlation, difference imaging, deconvolution, adaptative filtering, neural networks, Kalman filtering, expectation maximization, tomographic reconstruction, time reversal mirrors, synthetic aperture focusing, array phasing or in general any deterministic or stochastic signal processing method which allows for detection and characterization of defects.

The data processing results are represented as ultrasound images UI for specific bonding planes (e.g. B1, B2, B3) and adjacent timber lamellas, in which timber defects (D1, D2) are shown. Scanned trajectories and imaged timber defect areas are related, for example, with simple geometric relations or advanced tomographic transformations.

A direct ultrasound wave propagation model DPM can be used to define the incident parameters of transmitter and receiver. After input of the known parameters as width W, lamella height HL, sound velocity in the material of the laminated S, possible wave propagation paths through a laminated structure S can be computed. After such a simulation computation the settings of the transducer T, R can be adjusted and optimized.

The direct ultrasound wave propagation model DPM is used to derive the ultrasound propagation paths within the glued timber sample S for a specific transmitter configuration, from which configuration parameters for transducer T, R and associated electronics are obtained, and from which acquired wave signatures are correlated with type, position and geometry of timber defects D. In the simplest assumption a defect free laminated structure S is simulated with the direct ultrasound wave propagation model DPM and adequate setup parameters for the measurement are derived and adjusted.

The refraction of ultrasonic waves in discontinuities air-sample is modeled in a first approximation by applying Snell's law, which states

c₂ sin θ₁=c₁ sin θ₂ (1)

where c₁ and c₂ are the sound velocities for the media where the wave propagates initially and after the refraction respectively, θ₁ and θ₂ are the incident and refracted angles measured with respect to the normal of the discontinuity surface. It is typically assumed, that longitudinal waves are coupled into the sample. The reflection of ultrasonic waves is modeled with equal incident and reflected angles measured with respect to the normal of the discontinuity surface. A more involved analysis considers the anisotropic and heterogeneous properties of the laminated structure S to compute propagation paths for all mechanical waves generated at the discontinuity surface.

For laminated timber structure S inspection the material attenuation in wood increases with the frequency (23 dB cm⁻¹ MHz⁻¹), which reduces the usable frequency range to typically >20 kHz to 250 kHz. The center excitation frequency f of the transducer T is typically chosen to achieve a smaller wavelength than the mean timber lamella thickness HL.

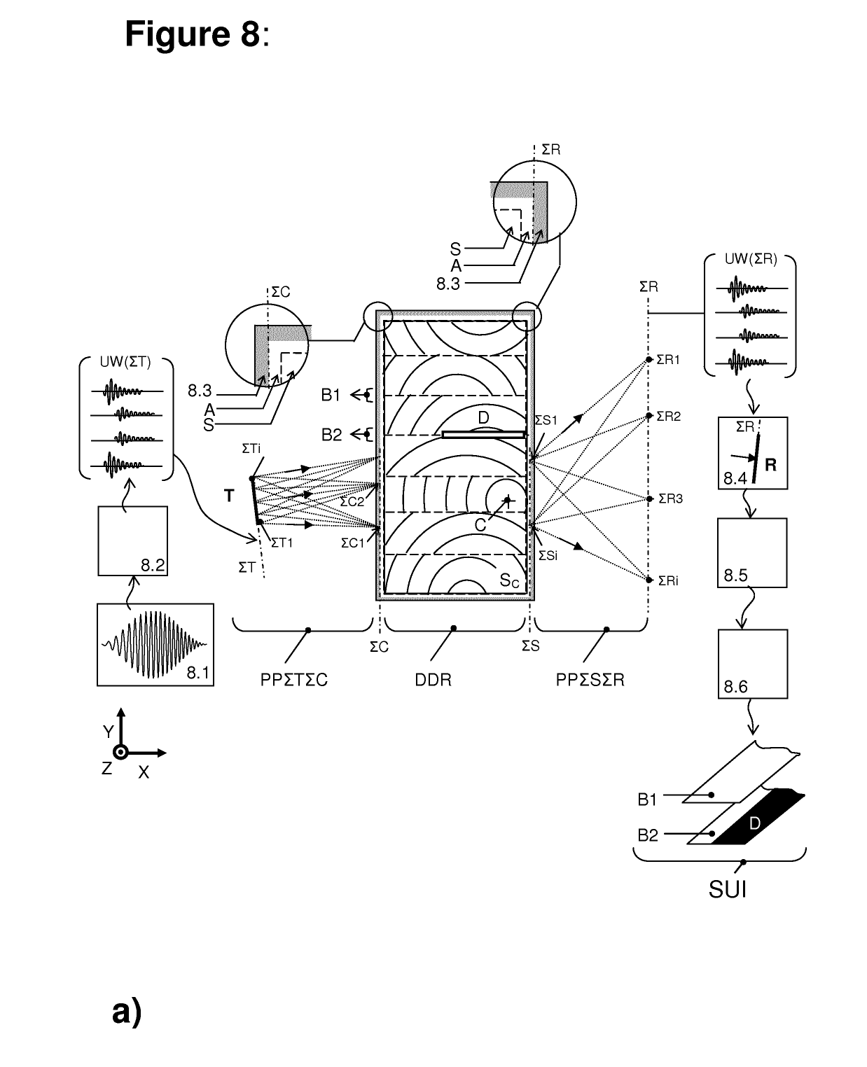

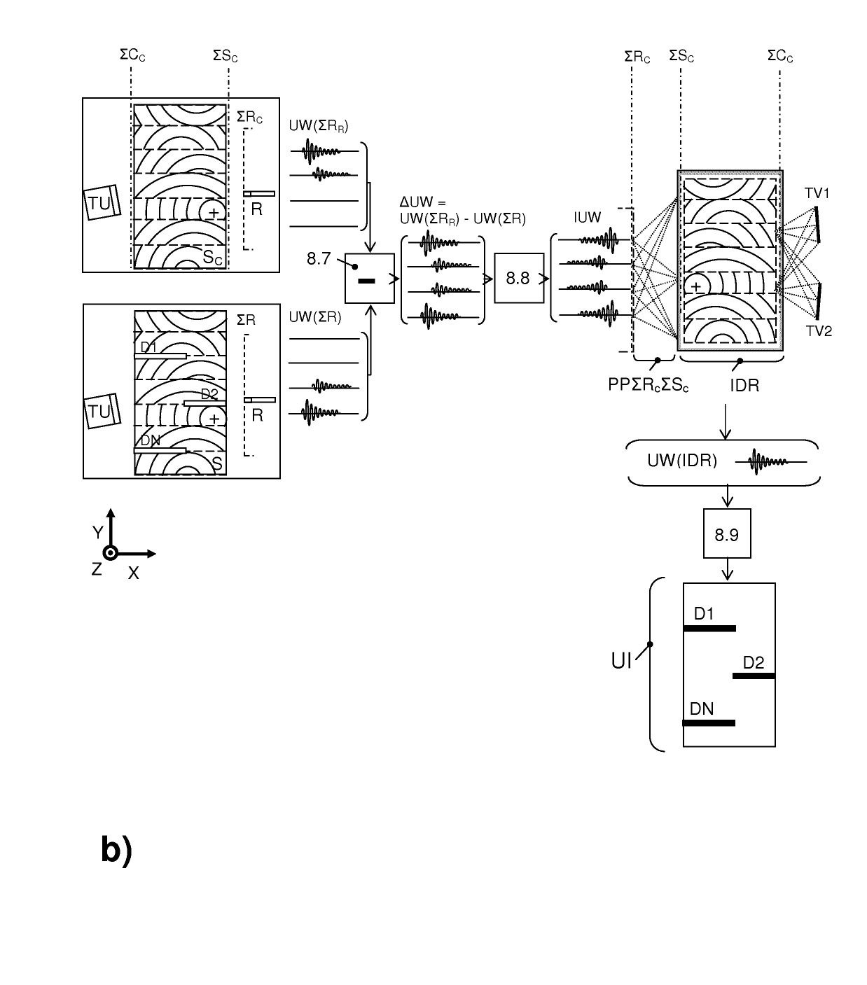

An inverse model IPM uses receiver data to invert inner wave propagation paths within the sample and to localize and characterize defects in the sample S. A specific implementation of IPM is described in detail in FIG. 8b.

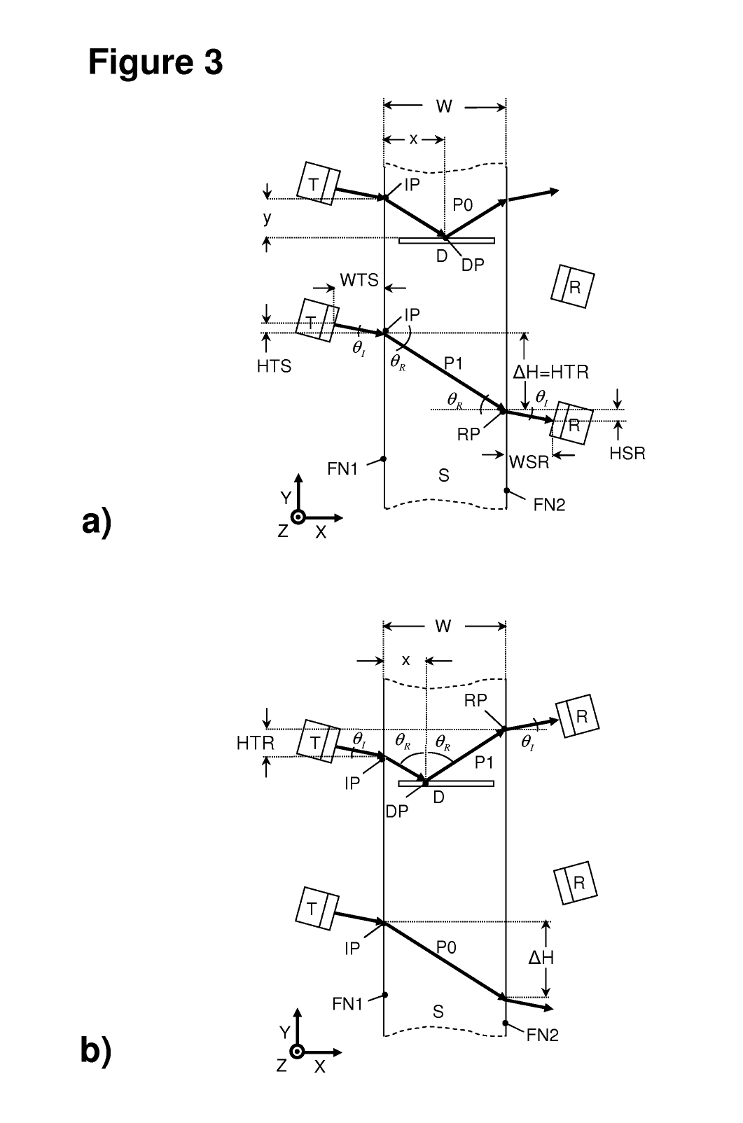

### FIG. 3

The relative position and orientation of the ultrasound receiver R with respect to the ultrasound transmitter T and the glued timber sample S determines which wave signatures are used in the setup and according to the inventive method.

A negative detection associates a decrease of ultrasound energy at the ultrasound receiver R of FIG. 3a to the presence of a defect D, as it is as well the case in the setup with T1 and R2 or T2 and R3 in FIG. 2. In this context, P1 are defined as ultrasound beam propagation paths which are substantially captured by the ultrasound receiver R, for instance the path from T1 to R1 or from T2 to R3 in FIG. 2, P0 are propagation paths which significantly diverge in position and/or orientation from the receiver R and are therefore not captured, for instance from T1 to R2 in FIG. 2. In the case of negative detection P0 is associated to a defect, P1 to no defects.

A positive detection associates an increase of ultrasonic energy at the ultrasound receiver R of FIG. 3b to the presence of a defect, as it is as well the case in the setup with transducers T1 and R1 in FIG. 2. In the case of positive detection, P1 indicates the presence of a defect, P0 corresponds to no defect.

In FIG. 3a a double-sided ultrasound inspection setup is disclosed, for which the two different lateral faces FN1, FN2 of the sample S are necessary to couple and read ultrasound energy. In FIG. 3a a specific section of S is shown in which a defect area D essentially perpendicular to faces FN1 and FN2 is assessed with negative detection.

The lateral faces FN1, FN2 are opposite faces of the glued timber sample S and are parallel to each other and separated by the sample width W. The rest of the dimensions of the specimen may have arbitrary extension and topology. The ultrasound beam IF generated by the transmitter transducer T is tilted with a small inclination θ_{I} from the normal of the lateral face FN1, the same inclination together with mirroring in X and Y is typically applied to the receiver transducer R. θ_{R} is the angle of refraction inside the glued timber sample, ΔH is the Y displacement of the refracted beam RF between FN1 and FN2 in defect-free regions of S, WTS and WSR are separations in X between glued timber sample and transmitter T and receiver R transducers, respectively, HTS and HSR are displacements in Y of the ultrasound beam in air for the transmitter and receiver transducers, respectively. HTR is the Y-displacement between the insonification/incident IP position and the re-radiation position RP.

For the setup of FIG. 3a, HTR=ΔH.

Applying Eq 1 and trigonometric derivations θ_{I}, θ_{R}, HTS and HSR are estimated from ΔH, WTS and WSR:

$$\begin{matrix}{θ}_{I} = arcsin ( \frac{{c}_{A}}{{c}_{S}} sin {θ}_{R} ) \\ = arcsin ( \frac{{c}_{A}}{{c}_{S}} sin ( arctan \frac{Δ H}{W} ) )\end{matrix} \\ HTS = WTS tan {θ}_{I} HSR = WSR tan {θ}_{I} \qquad ( 2 )$$

where c_{A} is the sound speed in air and c_{S} is the sound speed in the sample S. Typically c_{S}>c_{A} Implying that θ_{R}>θ_{I}, which allows an efficient Interaction of the ultrasound beam with defect areas for small θ_{I} values.

If T and R are scanned as a fix unit in Y the state of a defect area D, e.g. a bonding plane, is imaged at specific positions DP, separated by an X-displacement x from lateral Face FN1, by adjusting the Y-displacement y of the insonification/incident position IP with respect to DP:

$$y = x \frac{Δ H}{W} \qquad ( 3 )$$

FIG. 3b is an extension of FIG. 3a showing positive detection. The ultrasound beam generated by the transmitter transducer T is tilted with a small inclination θ_{I} from the normal of FN1, the same inclination together with mirroring in X is typically applied to the receiver transducer R.

The optimum Y-displacement HTR between the incident position IP and the re-radiation position RP for the P1 path is estimated for the setup of FIG. 3b as a function of the specific x coordinate of the defect position DP inspected:

$$HTR = Δ H ( 1 - \frac{2 x}{W} ) \qquad ( 4 )$$

In particular HTR=0 for inspection at the center of the sample width. FIGS. 3a-b may be generalized for lateral face FN1 and lateral face FN2 parallel or not parallel, opposite or not opposite, curved or not curved and/or for D curved.

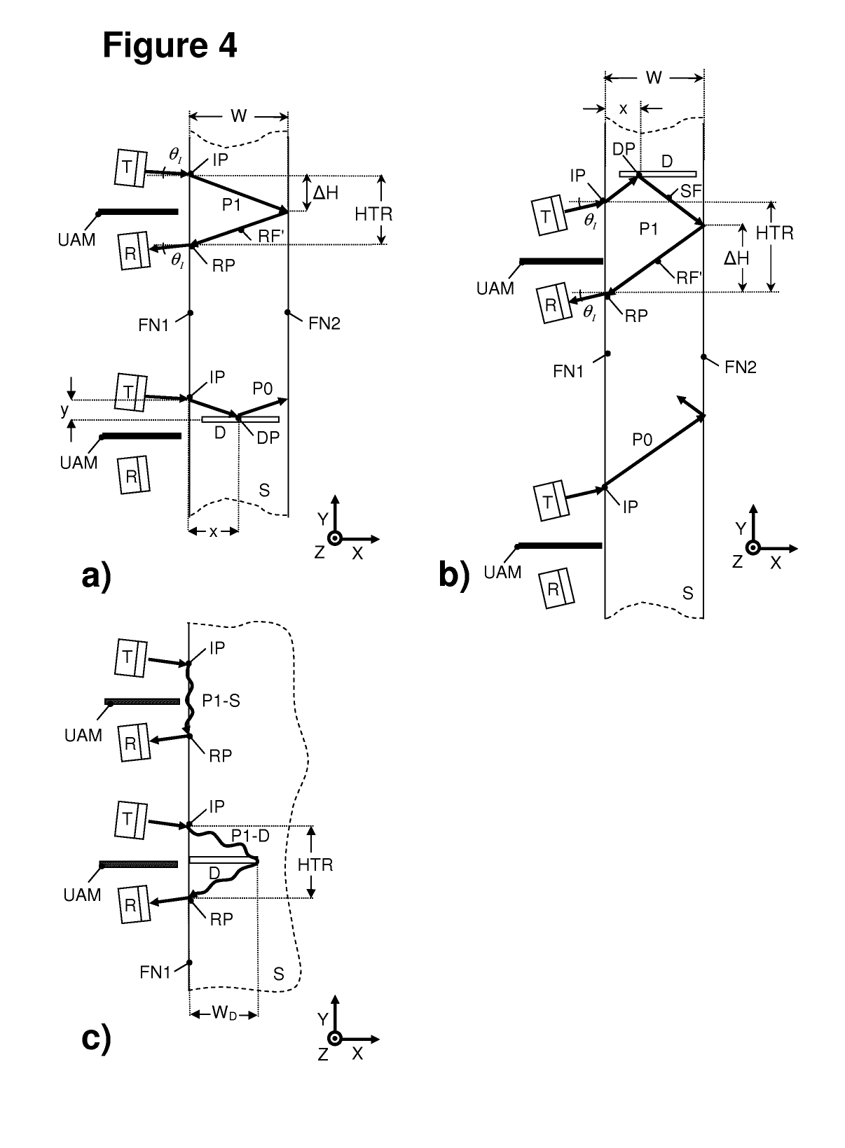

### FIG. 4

In FIG. 4, a single-sided inspection setup is defined, for which the ultrasound energy is coupled and re-radiated from the same lateral face FN1 of the sample S.

In FIG. 4a a specific section of the laminated structure S is shown in which a defect area D essentially perpendicular to the lateral face FN1 is assessed with negative detection. The propagation path P1 consists of an incident sound field IF, an ultrasound beam reflected at opposing lateral face FN2 RF′ and a re-radiated air path RRF. A barrier UAM from a highly attenuating material, a porous material or multiple delaminated material acts as blocking medium to avoid direct propagation of ultrasound energy through air between the transducers T, R.

The transducer positioning parameters are estimated in a similar fashion to FIG. 3. If lateral face FN1 is parallel to opposing lateral face FN2 Eq 2 and Eq 3 can be applied and HTR=2·ΔH. The rest of the dimensions of the specimen may have arbitrary extension or topology.

FIG. 4b is an extension of FIG. 4a for positive detection. The propagation path P1 consists here of a double reflection in the lateral back face of the sample FN2 and in the defect area D. In the particular case of FN1 parallel to FN2, HTR is estimated for the setup of FIG. 4b as a function of x with:

$$HTR = 2 Δ H ( 1 - \frac{x}{W} ) \qquad ( 5 )$$

In another single-sided inspection application, which is shown in FIG. 4c, a planar defect perpendicular to lateral face FN1 penetrates from the surface a certain depth W_{D} into the test object. Every sample face apart from lateral face FN1 has arbitrary dimensions or extension. Variations in wave signatures between a wave path P1−D diffracted around the defect and a wave path propagated directly through sample S between transmitter and receiver P1−S give information about the presence and geometry of the defect D. For example, the difference in time of arrival ΔTOA between P1−D and P1−S can be used to determine the width of the defect W_{D} by using in a first approximation the following expression:

$${W}_{D} = \frac{1}{2} \sqrt{{{c}_{P 1 - D}^{2} ( Δ TOA + \frac{HTR}{{c}_{P 1 - S}} )}^{2} - {HTR}^{2}} \qquad ( 6 )$$

where c_{P1-D} and c_{P1-S} are the mean sound velocities of the wave paths P1−D and P1−S, respectively. FIGS. 4a-c may be generalized for lateral face FN1 and lateral face FN2 parallel or not parallel, opposite or not opposite, curved or not curved, and/or for D curved.

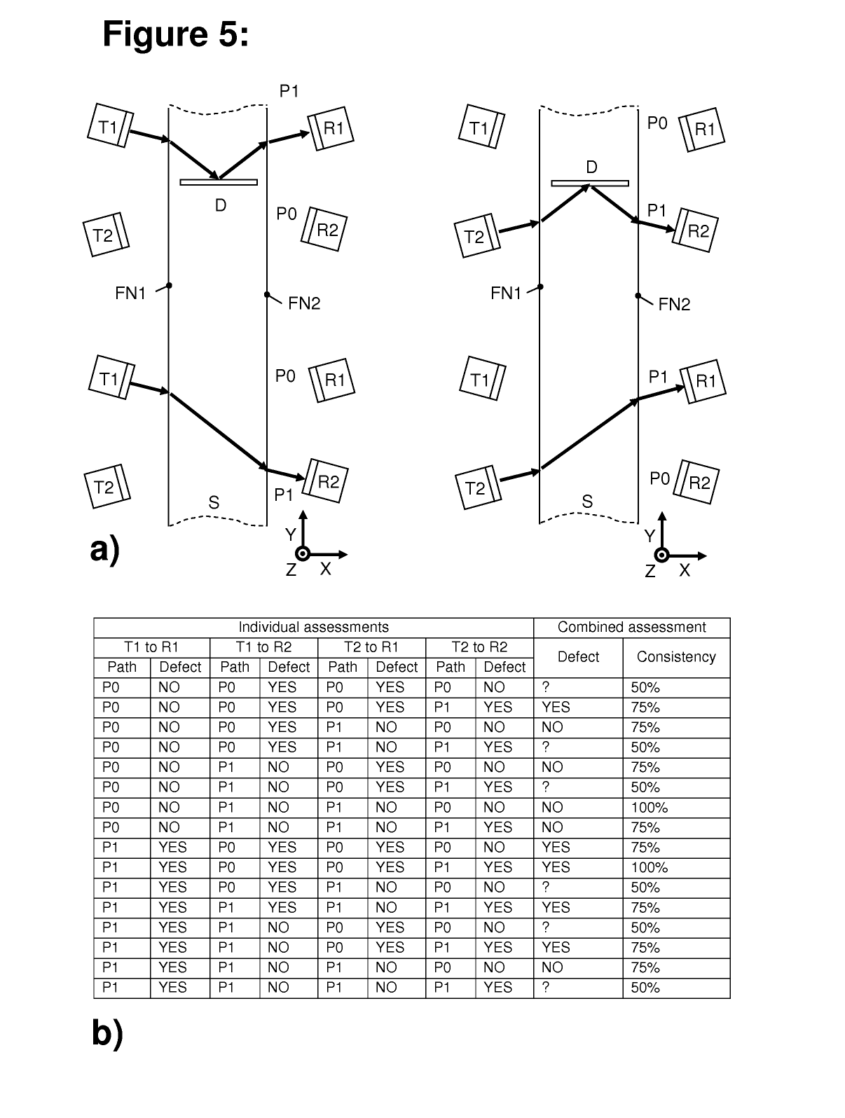

### FIG. 5

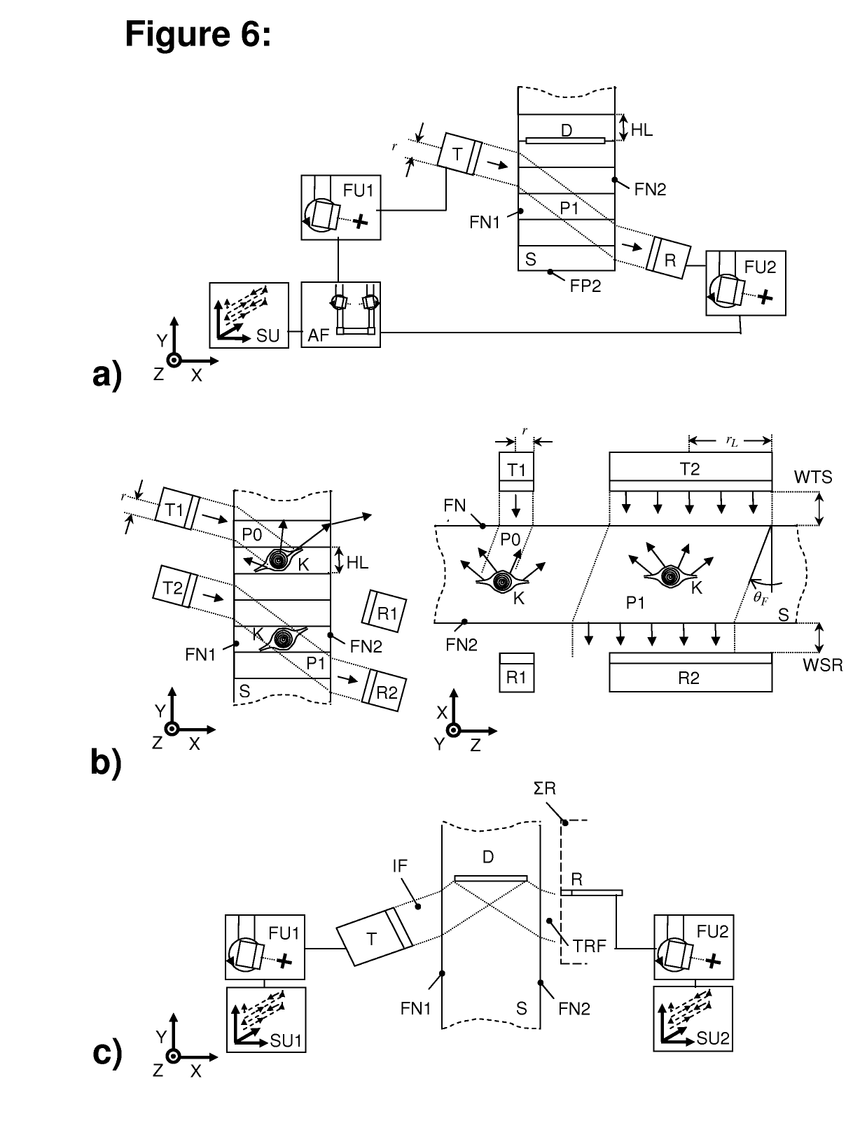

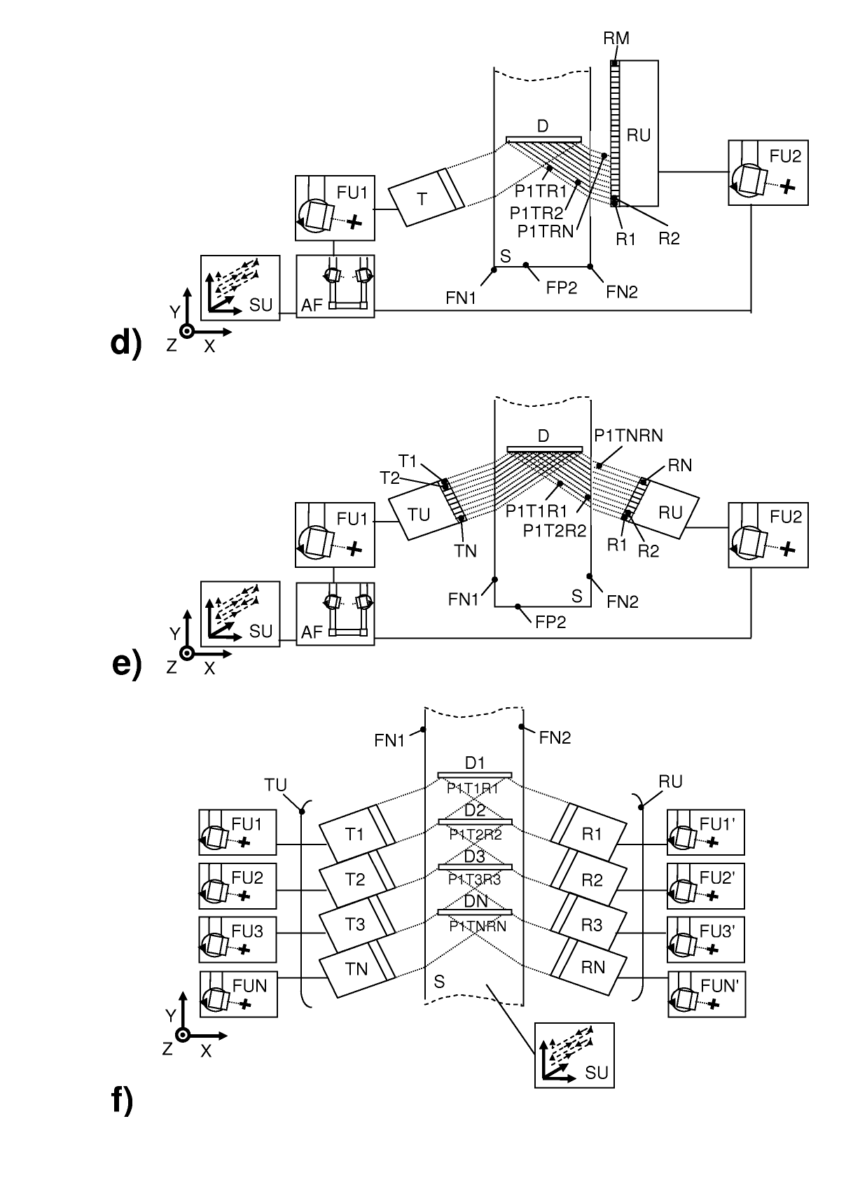

In FIG. 5 multiple setups of FIG. 3 and FIG. 4 are combined in a space diversity setup that provides simultaneously multiple observations of a single timber defect area D. Each setup couples ultrasound energy in a differentiated propagation path by combining positive and negative detection, by using specific transducer positions/orientations and/or by exchanging transmitter and receiver positions. The combination of multiple observations is used to improve defect characterization and differentiation with respect to the natural variability of the sample, such as, in the case of glued laminated timber beams, density, attenuation and sound velocity uncertainties, knots (K) and ultrasound energy shifts θ_{F} due to grain and ring misalignments (FIG. 6b).

FIG. 5a is a space diversity example in which four propagation paths (T1 to R1, T1 to R2, T2 to R1, T2 to R2) are tested. FIG. 5b shows a combination logic example for FIG. 5a. An individual assessment of timber defect presence (YES or NO) is obtained for each individual propagation path. A combined assessment is obtained in this case by a majority rule, and its consistency is calculated as the percentage of agreeing paths. In a more general implementation, specific ultrasound signatures, probability distributions, sensitivity and spatial resolution associated to each propagation path are used in the combination.

### FIG. 6

In FIG. 6, several possible implementations of the ultrasound excitation unit TU and ultrasound sensor unit RU with specific air-coupled ultrasound transducer assemblies are described, which are applied to the ultrasound inspection setups described in this application.

The simplest configuration, which is shown in FIG. 6a, consists of two transducers, namely one transmitter T and one receiver R, oriented and positioned with two fixtures FU1 and FU2, joined together with an attachment frame AF, and scanned as a single unit with a mechanical scanning system SU in order to couple and read an ultrasound beam at discrete positions and orientations of a glued laminated sample S. For such a setup, T is typically implemented with a planar transducer, which provides a well collimated ultrasound beam along the full wave propagation path P1, and is usually described with a baffled piston model. The lateral resolution of the sound field generated by a single transducer is limited by a minimum half power beamwidth of r/2, where r is the principal radius of the active area of the transducer. The distance WTS between transmitter transducer and sample typically satisfies WTS>0.5r²c_{A}⁻¹f, where f is the center excitation frequency, in order to avoid near field oscillations and achieve a well-collimated sound field. Focused transducers may as well be used to improve the lateral resolution down to a lower bound given by the wavelength in the sample λ_{S}=c_{S}/f. A good directivity of the ultrasound beam is generally wished in order to limit the number of propagation paths coupled into the sample, the directivity increases with the ratio r/λ_{A}, with λ_{A} the wavelength in air. The ultrasound energy coupled into the sample increases with r⁴. For glulam inspection a good compromise between resolution, coupling and directivity is typically reached for r=0.5 HL, where HL is the mean lamella height.

Local heterogeneities due to the natural variability of the sample such as, in the case of timber, knots K, ultrasound energy flux shifts θ_{F} due to ring and grain misalignments or density, attenuation and sound velocity uncertainties, can lead to significant sound field scattering and to non-captured propagation paths P0 for regions of the sample in which no defects are present, as it is the case for transducers T1 and R1 in FIG. 6b. Rectangular transducers elongated r_{L} along the length direction of the beam Z, e.g. T2 and R2, may be used to reduce the influence of local heterogeneities in the propagation path P1, which is then only blocked by long planar defects, such as lamination flaws. The lateral resolution of the sound field then limited by r_{L}/2 in Z and by r/2 in Y.

The receiver R is typically Implemented with a transducer of similar dimensions to the transmitter T, in order to efficiently capture the ultrasound energy coupled into the sample. A directive receiver (large r/λ_{A} ratio) is typically used to select only waves propagating in a specific orientation from the total sound field re-radiated from the sample and to filter out undesired wave propagation paths from the acquired ultrasound signals. The distance WSR between receiver transducer and sample is typically chosen for a pulsed excitation system so as to separate in time multiple reflections between receiver transducer and sample surfaces, which is achieved with WSR>0.5c_{A}T_{R}, with T_{R} the length of the analyzed time window in the received ultrasound signals.

In another setup shown in FIG. 6c, the receiver transducer R is realized with an element of small active area, typically a calibrated microphone which, for a fixed position and orientation of the transmitter T, is scanned in a three-dimensional region ΣR by means of a fixture FU2 and a scanning unit SU2. Ultrasound signals are measured for all scanned positions of ΣR and stored as a multiplicity of ultrasound waveforms UW for further data analysis. The procedure is repeated for specific positions and orientations of T, which are adjusted independently from R by means of a fixture FU1 and a scanning unit SU1. For a transducer R with r/λ_{A}<1 a quasi-omnidirectional detector is obtained, which allows recording of the total re-radiated sound field TRF in air by sample S, which is the sum of RRF and RSF contributions for a specific incident sound field IF. The information recorded is then used as input to the inverse propagation model IPM in order to invert the wave propagation paths within the sample and the sound field distribution at defect areas D, from where defects are identified and characterized.

The setup depicted in FIG. 6d, shows the use of a multi-element receiver sensor unit RU. The individual transducer elements R1, R2, . . . RM may be arranged as an ultrasound array, e.g. 1D or 2D, or as non-uniformly distributed sensors within some spatial domain. If the sensors are not omnidirectional, some tilting of the receiver transducer is recommended in a preferred reception direction. The signals received from individual transducer elements are typically filtered and combined together in a single output or recorded individually using a multiplexing system (typically time multiplexing at sampling or pulse repetition rates) or multiple acquisition channels. One typical application of such a system is fast acquisition of the re-radiated sound field, in which the mechanical scanning performed by SU2 in FIG. 6c is replaced by electronic scanning of the signals captured by receivers R1, R2, . . . RM in FIG. 6d. Each transducer element in FIG. 6d then provides a reading equivalent to a single scan position of R in FIG. 6c. The transmitter T and the multi-element receiver unit RU can be scanned with a single unit SU as in FIG. 6a. Specific combination laws, e.g. phasing and apodization, for received signals allow, among others, for synthesis of a directive receiver with tunable orientation, filtering out of spurious signals and improvement of lateral resolution. Individual propagation paths, e.g. P1TR1, P1TR2, . . . , P1TRN, can be selected for a specific ultrasound beam excitation, so that electronic scanning of defect areas D is achieved. Typically multiplexing and low-noise electronics are implemented within the casing of the associated transducer element A careful routing, isolation and alignment is required for each transducer element R1, R2, . . . RM.

FIG. 6e shows a generalization of FIG. 6d, in which the transmitter is as well implemented with a multi-element excitation unit TU. The individual transducer elements T1, T2, . . . TN may be directive or omnidirectional and are arranged in a specific spatial configuration, similarly to the receiver unit RU. The transmitter elements are driven either all with the same electrical signal or each with individual excitations. For example, a common excitation signal may be introduced with specific phasing and scaling factors for specific sensors in order to achieve a directive ultrasound beam of tunable position and orientation, moreover focused at specific spatial positions. In general, a specific sound field distribution can be synthesized at the insonified lateral face FN1 of the sample S, which propagation through the sample S leads to high interaction with only certain defect types and geometries. Another possibility is to couple individual wave propagation paths P1T1R1, P1T2R2 . . . P1TNRN into the sample S and individually record specific sound field portions of the re-radiated sound field, so that electronic scanning of defect areas D is achieved.

FIG. 6f shows a specific implementation with several transmitter and receiver transducer pairs (e.g. T1-R1, T2-R2, T3-R3, . . . TN-RN) being used for coupling and acquisition of propagation paths P1T1R1, P1T2R2, P1T3R3, . . . P1TNRN, which allow for simultaneous inspection of multiple defect areas D1, D2, . . . , DN; for example, individual laminations of a glued laminated structure S. Each individual transmitter and receiver transducers can be in turn implemented as a single or multi-element transducer. Such an implementation is attractive for a system in which mechanical scanning is implemented only along Z, for example by scanning the sample with a conveyor belt SU for quality control during production of glued laminates.

In a more general implementation, each receiver element R is implemented with specific sensor technologies which are capable to detect the sound field re-radiated through the sample and for which there Is no contact between receiver elements R and sample S.

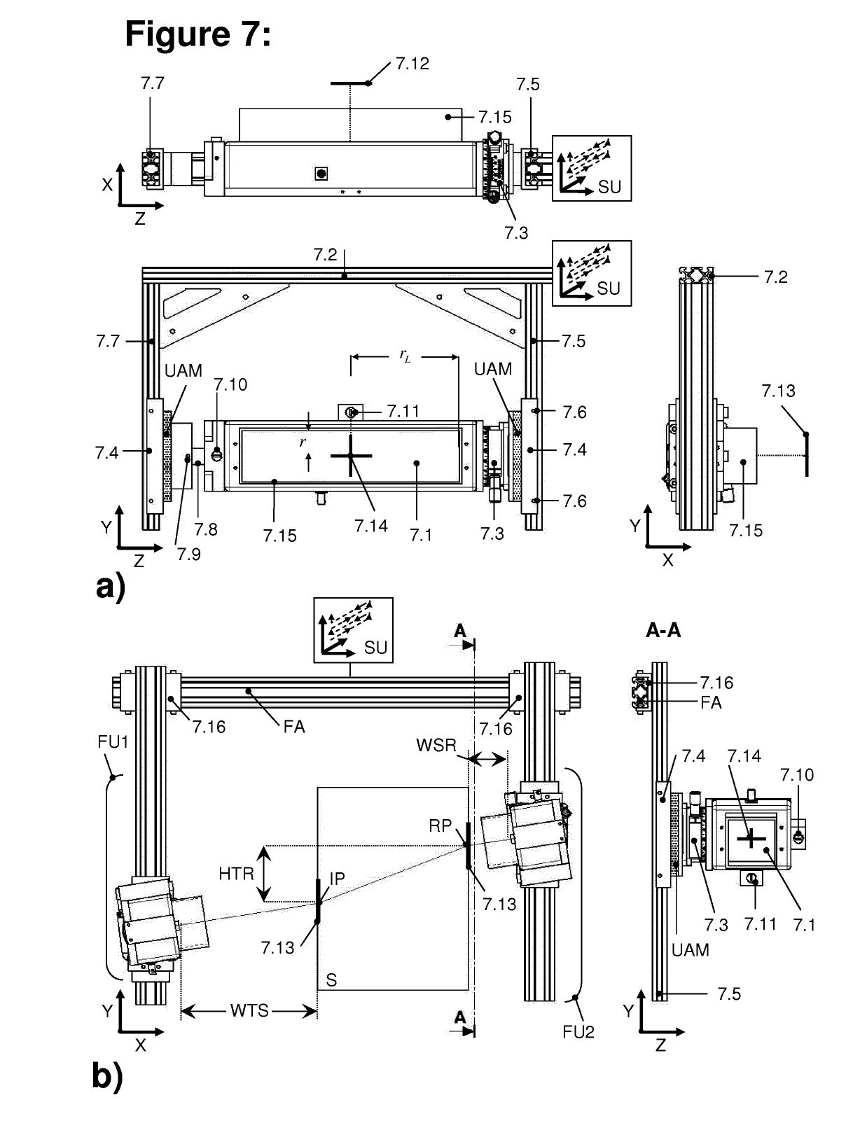

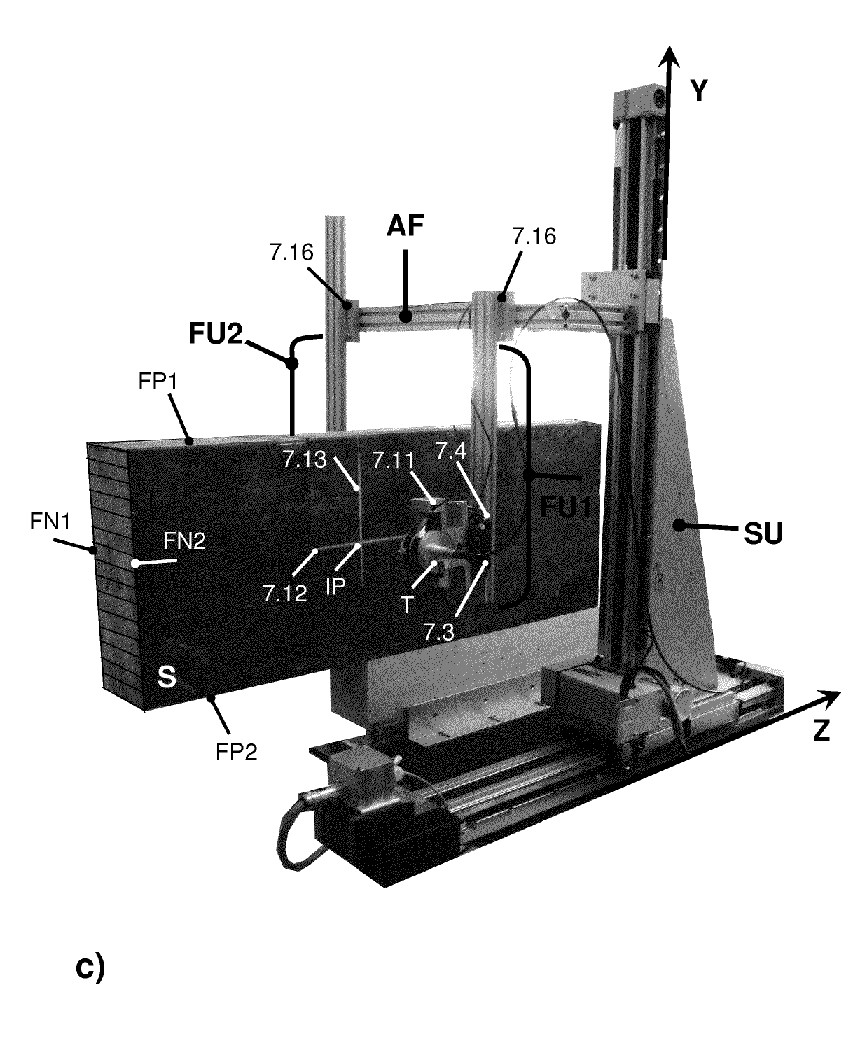

### FIG. 7

A specific realization of a mechanical construction is described in FIG. 7, which allows for transducer orientation, positioning and scanning for specific setups described in the invention. This mechanical construction allows pivoting and positioning of a transducer T, R to be used in the inspection setups.

FIG. 7a shows a specific implementation of a transducer positioning fixture FU for an elongated transducer as described in FIG. 6b. The fixture allows adjustment of the angle of insonification θ_{I}, and Y-displacement HTR between insonification position IP and re-radiation positions RP, providing moreover a positioning reference for both IP and RP. The transducer 7.1 may be a transmitter T or a receiver R, single element or multi-element. The fixture may remain static or be attached to a scanning unit SU through the mounting profile 7.2, which is typically implemented as a hollow aluminum profile with rails for screw attachment. A precision rotary stage 7.3 is used to control the inclination of the transducer, which in turn equals θ_{I}. Instead of a precision rotary stage 7.3 other mechanical components can be used, e.g. goniometers or calibrated wedges, which allow for reproducible rotation and fixation. The inclination angle may be adjusted manually or with a motorized system.

The barrier UAM is a porous or multiple layered material through which ultrasound waves are highly attenuated, which prevents coupling of ultrasound waves between transmitter and receiver transducers when their transducer positioning fixtures FU are mechanically connected as e.g. in FIG. 7b. The same material may be used at other positions of the fixture.

A U-shaped plate 7.4 connects the transducer to the rails of mounting profile 7.5 at an arbitrary Y position by adjusting the lateral screws 7.6. An additional mounting profile 7.7 may be used in order to mechanically stabilize an elongated transducer, together with the barrier UAM, the U-shaped plate 7.4 and a cylindrical shaft 7.8, which allows free rotation of the transducer 7.1 according to precision rotary stage 7.3 unless fixed with a screw 7.9. The U-shaped plate 7.4 may be replaced by a motorized linear stage.

A targeting device is formed by a first line laser 7.10 and a second line laser 7.11 which are able to generate a light cross 7.14 on the insonified lateral face of the glued timber sample at exactly the point of incidence of the ultrasound beam, which significantly simplifies transducer positioning with respect to the sample. Horizontal 7.12 and vertical 7.13 light lines are forming the light cross 7.14. The light cross 7.14 accurately marks the insonification position IP for a transmitter transducer T and the re-radiation position RP for a receiver transducer R. Thus 7.10 and 7.11 provide a simple and reliable position reference of the transducer with respect to the sample for arbitrary transducer orientation and position.

A tubular structure 7.15 around the transducer is used to avoid coupling or acquisition of ultrasound energy in orientations divergent from the main ultrasound beam e.g. beam side lobes. Additional items may be added to the mounting profiles 7.2, 7.5, 7.7 to e.g. adjust the azimuth of transducer 7.1.

FIG. 7b shows a specific mechanical construction which can be used to implement the double-sided inspection setups of FIG. 3. The construction may remain fixed or be attached to a scanning unit SU. Two transducer positioning fixtures FU1 and FU2, which are simplified with respect to the one described in FIG. 7a, are used for transmitter T and receiver R transducers, respectively, each adjusted to achieve specific θ_{I}, HTR, and insonification IP and re-radiation RP positions with respect to sample S. The two fixtures FU1 are mechanically connected as a single unit through an attachment frame AF, which is implemented with a mounting profile of the same kind as 7.2, 7.5 or 7.7. U-shaped plates 7.16, similar to 7.4, are used to connect AF to FU1 and FU2 and allow calibration of the X-displacements WTS and WSR between transducers T, R and sample S. 7.18 may be replaced by a motorized linear stage. The single-sided inspection setups of FIG. 4 can also be implemented with the setup of FIG. 7b by attaching the transmitter T and receiver R to the same mounting profile 7.5.

The positioning and orientation of the transducers are mainly adjusted with simple calibration procedures: for example, the transducers may be placed in a configuration where a maximum of ultrasound energy is expected (positive detection) and the angles of the goniometer and rotary stage are adjusted until a transmission maximum is effectively reached (best alignment position). The same calibration procedure may be implemented for an absolute minimum search in the case of negative detection.

FIG. 7c illustrates a specific implementation of a mechanical system according to the setup of FIG. 6a. A similar construction to the one described in FIG. 7b is moved as a fixed unit along the height and length of a glued laminated sample S, specifically a glued laminated timber beam, by means of a computer-controlled two-axis mechanical scanner SU, which implements continuous scanning in Y and Z. The scanning is typically performed in a raster fashion with respect to a rectangular area of the lateral faces FN1, FN2 of the sample S. HTS, HSR, HTR and θ_{I} are fixed during the scan. The scanning steps can be set well-below the wavelength in the sample λ_{S}. Noise reduction in the digitized ultrasound waveforms UW can then be achieved by considering waveforms recorded at adjacent scanned pixels as differentially modified repetitions of the same signal, from where noise statistics can be calculated and filtered out. If for example the rotary stage 7.3 and the U-shaped connection plate for HTR calibration 7.4 and HSR calibration 7.16 are implemented with motorized units, then the relative positioning and orientation of the transducers can be tuned for each scanned position, otherwise they are fixed during the scan.

The system illustrated in FIG. 7c can be generalized by using non-connected transducer fixtures FU1 and FU2, attached to independent scanning units SU1 and SU2. FU1 and FU2 may then be moved synchronously, in which case the setup is equivalent to FIG. 7c with the difference that no attachment frame AF is required. Another option is to perform scans of FU2 for specific FU1 positions, which implements the setup of FIG. 6c. FU1 and FU2 are typically scanned in a plane containing Y and Z directions as in FIG. 7c, volumetric scans or scans along curved trajectories are also possible. Electrical scanning and mechanical scanning can be combined, as it is shown e.g. in FIG. 6d and FIG. 6e. The mechanical or electric scanning data is recorded in ultrasound spatial datasets, e.g. lists of time waveforms for specific scanned positions or transmitter-receiver combinations, from which ultrasound imaging of specific defect areas and tomographic information of the internal state of the sample are obtained. Another option consists of moving the inspected sample with respect to fixed ultrasound transducers, for instance with a conveyor belt, as it is shown e.g. in FIG. 6e.

### FIG. 8

In FIG. 8, specific ultrasound wave propagation models are detailed, which describe the generation and acquisition of ultrasound waves and their coupling and interaction with a glued timber sample S containing specific defect areas, from which, in combination with experimental data, defects are localized and characterized, and specific parameters of the ultrasound inspection method and installation described by the invention are optimized.

A direct ultrasound wave propagation model DPM is specified for a calibration glued laminated sample S_{C}, for which material properties and defect D positions and geometries are known a priori, and for which ultrasound incident sound fields are defined by single or multiple element ultrasound transducers T. The model DPM calculates the sound field at specific regions of the sample S_{C} or re-radiated into air and simulates ultrasound signal acquisition and processing.

According to a specific implementation, which is shown in FIG. 8a, a model 8.1 of an arbitrary waveform shape generated by waveform generator 2.2, typically pulsed, is filtered with a transfer function 8.2 which describes the gain, delay and distortion introduced by the electrical equipment 2.3 and transmitter transducer T. The strain and stress fields at the radiation surfaces of the transducer ΣT are calculated with a theoretical model, e.g. baffled piston, or by experimentally determining the pressure field distribution in the near field of the transducer. The sound field in ΣT is represented with a list of ultrasound waveforms UW(ΣT), each representing an elementary ultrasound point source at discrete pixels ΣT1, ΣT2 . . . ΣTi of ΣT. The incident air pressure field IF distribution is represented by a list of ultrasound waveforms UW(ΣC), typically shown as amplitude vs. time diagrams, in a plane ΣC in the vicinity of S_{C}, typically parallel to the insonified lateral face FN1, with the distance between ΣC and S_{C} being arbitrary small. UW(ΣC) is efficiently calculated from UW(ΣT) by means of a point source projection PPΣTΣC between ΣT and ΣC. Ultrasound waveforms UW for each pixel ΣCi of ΣC are computed with PPΣTΣC by filtering each ultrasound waveform UW in ΣTj with a transfer function that represents the ultrasound propagation between ΣTj and ΣCi, and by adding up the filtered contributions of ΣT1, ΣT2 . . . ΣTi in ΣCi.

A direct discretization region DDR is defined, in which the ultrasound coupling and wave propagation phenomena within the calibration sample S_{C} are analyzed. The input of DDR is an incident field sound IF represented by UW(ΣC). The output of DDR is the total re-radiated sound field TRF, which is the sum of RSF and RRF contributions, and is represented by a list of ultrasound waveforms UW(ΣS) in a plane ΣS in air in the vicinity of S_{C}, typically parallel to the re-radiation face (FN2 in FIG. 8a), the distance between ΣS and S_{C} being arbitrarily small. For the single-sided inspection setups of FIG. 4, ΣS is defined in the vicinity of FN1.

Typical discretization strategies for DDR are finite-difference or finite element methods or ray tracing models in time or frequency domain, which divide DDR in a defined number of voxels for which parameters (e.g. stiffness tensor, density, damping coefficients) of specific materials (air, defect D, sample S_{C} . . . ) are known a priori and elastic quantities (stress and strain) are computed. In the case of timber, additional models, e.g. cylindrical orthotropy with respect to year ring curvature centers Ci, can be used to incorporate for each voxel specific sample features such as ring, grain, knots, pith, sapwood, heartwood, local heterogeneities, specific timber defects or moisture content distribution. The direct discretization region DDR is typically limited by a thin air layer A, which allows for direct coupling of the incident sound field IF in ΣC and for direct acquisition of the total re-radiated sound field TRF in ΣS, and by radiating boundaries 8.3, e.g. absorbing boundary conditions or perfect matched layers, which eliminate undesired computational artifacts at the boundaries of DDR. The sound field calculated in a plane ΣS in air in the vicinity of the sample S_{C} is projected with a point source projection PPΣSΣR to a list of discrete receiver positions ΣR1, ΣR2, ΣR3 . . . ΣRN, which build up a receiver surface ΣR. The output is a list of simulated ultrasound waveforms UW(ΣR), similar to the measurement data obtained with the setup of FIG. 6c.

Specific finite diameter receivers 8.4 are modeled by choosing ΣR1, ΣR2, ΣR3 . . . ΣRN as a discrete grid of receiver positions over the active area ΣR of a receiver transducer R and by filtering and adding up their associated ultrasound waveforms UW(ΣRi). Specific transducers (omnidirectional, directive) with defined positions and orientations can be simulated with this method. Specific propagation paths, e.g. waves with specific orientation and/or polarization, are selected from the total sound field calculated in the sample and air by simulating directive receivers or by selection of ultrasound waveforms UW representing specific strain and stress components. The gain, delay and distortion introduced by the receiver transducer R and acquisition equipment are incorporated with a transfer function 8.5, in which noise and spurious signals may as well be included. The synthetic ultrasound data generated is finally processed with the same signal processing and defect imaging algorithms 8.6 used for experimental data, from where ultrasound images SUI of defects D in specific bonding planes B1, B2, . . . and lamellas are calculated. The simulated images SUI are compared with experimental data acquired for S_{C} with the method and installation described by the invention. The simulated ultrasound waveforms and images are used as input to optimization algorithms to calibrate the inspection setup, e.g. transducer positioning and orientation or optimum excitation signals, and to tune the data evaluation of ultrasound data.

A[[n]] specific inverse ultrasound wave propagation model IPM uses experimental ultrasound data recorded through a laminated structure S with a priori unknown defects D1 D2 . . . Di, experimental data recorded through a well-known calibration sample S_{C}, and a direct ultrasound wave propagation model of S_{C} in order to invert inner wave propagation paths within the sample S and to localize and characterize defects D1 D2 . . . Di in S.

In a specific implementation, which is shown in FIG. 8b, a sample S contains defects D1, D2 . . . Di, which number, position, type and geometry are a priori unknown and to be determined. A defect-free calibration sample S_{C} or a direct propagation model DPM that describes sample S is available. The material properties of S and S_{C} are equivalent except for the defects D1, D2 . . . Di. A calibration sample S_{C} can be e.g. obtained from a section across the length of S which is known to be defect-free. A model of S_{C} can be obtained with knowledge of material properties of defect free S from tabulated data (stiffness tensor, density, damping coefficients . . . ). The calibration sample S_{C} is excited with an excitation unit TU, consisting of single or multiple element ultrasound transducers T driven by specific excitation signals, and a list of ultrasound waveforms UW(ΣR_{C}), which describe the total sound field TRF re-radiated in air by sample S_{C}, are acquired at a surface ΣR_{C} with e.g. the experimental setup of FIG. 6c, or are simulated with a direct propagation model DPM as e.g. described in FIG. 8a.

The investigated sample S is then excited with the same excitation unit TU and a list of ultrasound waveforms UW(ΣR), which describe the total re-radiated sound field TRF re-radiated in air by sample S, are acquired at a surface ΣR with e.g. the experimental setup of FIG. 6c. A list of difference waveforms ΔUW is obtained by a subtraction 8.7 of each waveform in UW(ΣR) from its counterpart in UW(ΣR_{C}). The difference waveforms ΔUW are then subjected to a mirroring 8.8 with respect to their time axis. The calculated waveform list IUW is used as excitation of a mirrored propagation model of S_{C}. IUW is coupled through ΣR_{C}. The sound field in ΣS_{C} is calculated with a point source projection PPΣR_{C}ΣS_{C} similar to the ones described in FIG. 8a. Ultrasound coupling and wave propagation are simulated within an inverse discretization region IDR, in which the ultrasound waves are coupled through ΣS_{C}, back-propagated through the model of sample S_{C} and re-radiated through ΣC_{C}. IDR in FIG. 8b is implemented in a similar way to the direct discretization region DDR in FIG. 8a, except that coupling and re-radiation planes are exchanged and material parameters need to be transformed to compensate for damping in the direct propagation model. The ultrasound waveforms UW(IDR) calculated for each voxel of IDR are processed with specific signal processing algorithms 8.9 which extract specific wave signatures for each ultrasound waveform UW recorded. Typically ultrasound amplitude estimates at specific time windows T_{R}. are used. The wave signatures are plotted as ultrasound images UI of the interior of the sample S. With the described setup sound field convergences occur at the regions of IDR corresponding to unknown defects D1, D2 . . . DN of S, which allow for determination of defects in UI, for instance, in positions where amplitude maxima are observed. The procedure is repeated for a list of excitation units TU1, TU2, . . . TUi so that ultrasound wave interaction with all timber defect areas of interest is obtained, and the calculated ultrasound images UI are combined into a single defect map UI of sample S.

The described inversion method can be repeated iteratively, e.g. by alternatively insonifying ΣC_{C} or ΣS_{C} in IDR with the sound field recorded in each face in the previous iteration step. IDR may be substituted by an experimental configuration which is reciprocal with respect to the one used to measure UW(ΣR_{C}), and in which UW(ΣR_{C}) is synthesized as incidence field by a excitation unit TU and back-propagated in S_{C}, the sound field in ΣC_{C} being measured e.g. with the setup of FIG. 6c. Another possibility consists of setting ΔUW equal to UW(ΣR), calculating UW(ΣC_{C}) with IDR and letting the sound field in ΣC_{C} propagate in air, so that sound field convergences are achieved at specific virtual transmitter transducer TV1, TV2, . . . TVi positions and orientations. Divergences in position and orientation of TV1, TV2, . . . Ti with respect to the transducers used in the excitation unit TU are then associated to specific defects. Other sound field inversion techniques, such as time reversal mirrors, synthetic aperture focusing or tomographic reconstruction may as well be used.

All embodiments described in the invention are implemented for either single inspection or periodic monitoring of the sample under test. In the latter case, which is typically associated to in situ inspections at construction site, periodical measurements of the sample are analyzed; differences between successive measurements reveal changes in structure due to the development of timber defects. The reproducibility error of the air-coupled ultrasound method is below 1%, which allows tracking of small signal variations. The variations in temperature and moisture content in the sample and in air are estimated by an independent test method or an air coupled ultrasound self-calibration procedure based on direct air transmission measurements (no sample between transducers) or by monitoring mean signal changes for all scanned positions of the sample. This holds for any other variation not produced by timber defects which needs to be compensated.

### Example 1

### Negative Detection of Timber Defects

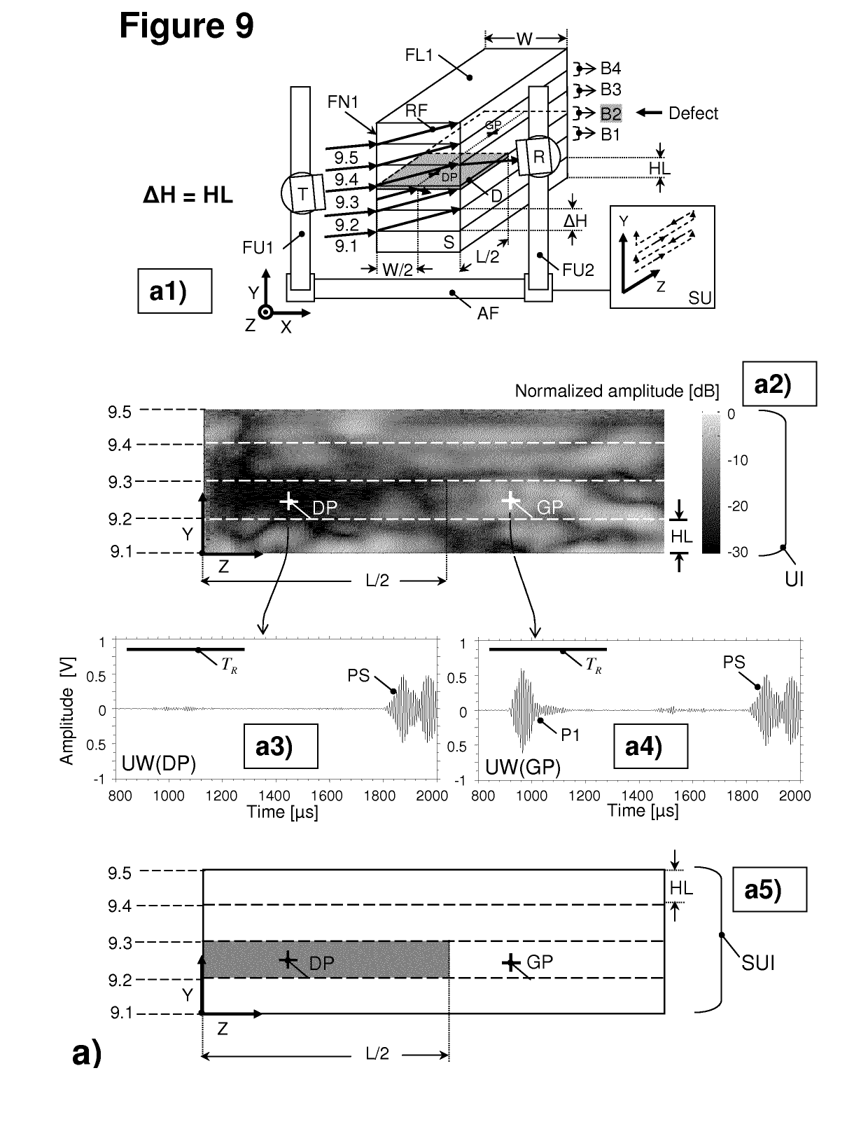

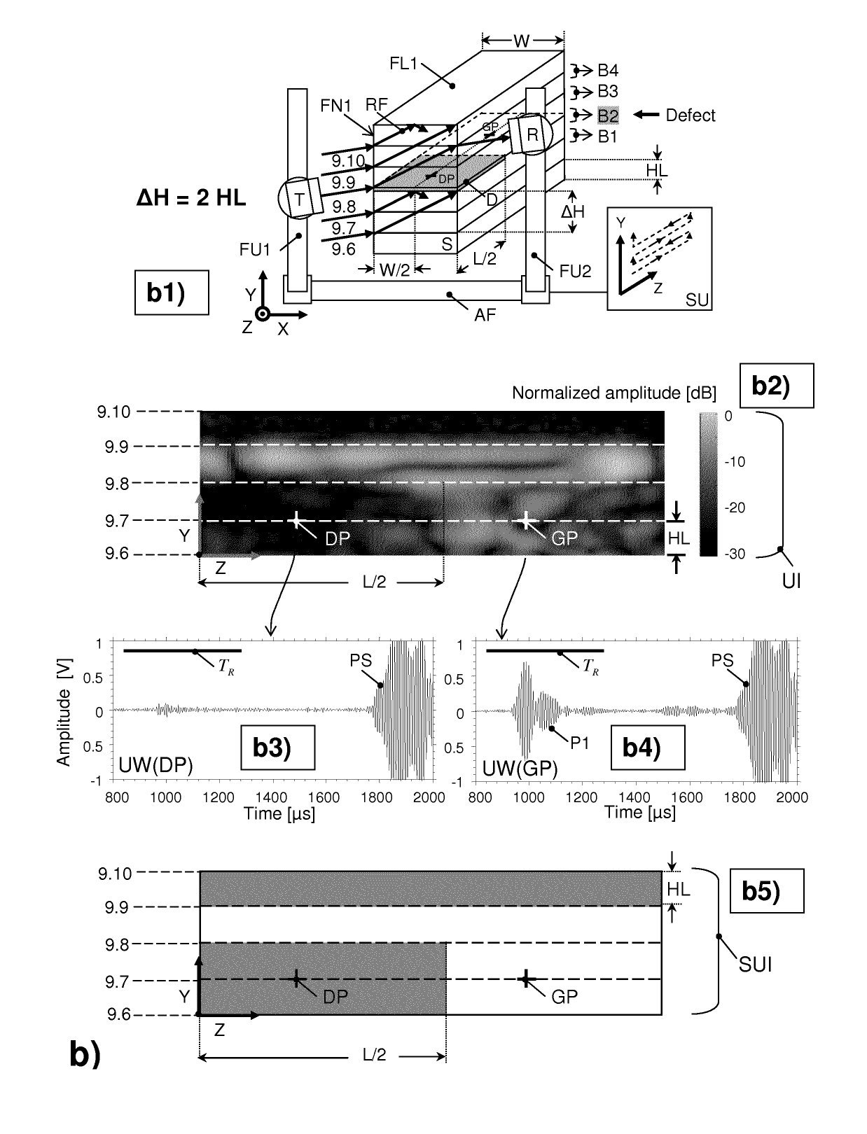

FIGS. 9a1 and 9b1 are showing two specific experimental implementations of an installation according to the negative detection ultrasound inspection setup described in FIG. 3a together with recorded ultrasound signals (FIGS. 9a3, 9a4, 9b3, 9b4) for a typical glued laminated timber beam and imaging results (FIGS. 9a3, 9b2) for individual bonding planes.

### Materials and Methods

The inspected sample S is a commercial glued laminated timber beam made from coniferous wood (Picea abies Karst.), which dimensions are L=500 mm, W=160 mm, H=245 mm. The mean height of the lamellas is HL=40 mm. A lamination defect D, specifically a non-glued region, was introduced in the bonding plane B2 for half of the beam length L/2 (160×250 mm²).

A single transmitter T and a single receiver transducer R were implemented with off-the-shelf gas matrix piezoelectric composites (The Ultran Group, Inc.) with a 30 dB bandwidth between 70 and 180 kHz. The transducers are planar and directive, with a radiation surface of circular shape and radius r=25 mm. The transducers were attached to two fixture units FU1, FU2, as described in FIG. 7a, FU1 and FU2 were joined with an attachment frame AF, as described in FIG. 7b, and scanned as a single unit with a two-axis mechanical scanner SU in Z and Y directions, as described in FIG. 7c, corresponding to the length L and height H of the sample S, respectively. The scanning was performed in a raster fashion with respect to a rectangular area of the lateral faces FN1, FN2. The scanning resolution was 1 mm in Y and 4 mm in Z.

The transducers were configured according to the negative detection setup of FIG. 3a; the angle of incidence θ_{I} and the relative positioning of both transducers were fixed for all scanned positions. Specific values were used for ΔH, namely the Y-displacement of the refracted beam RF between the lateral faces FN1 and FN2 at defect-free positions. In FIG. 9a11, ΔH=HL was chosen as the width of one timber lamella HL. In FIG. 9a2, ΔH=2 HL was chosen as the width of two timber lamellas. The remaining geometric parameters of this setup were computed with Eq 2 and are summarized in the following table:

| | | Values | |
|---|---|---|---|
| Parameter | Description | FIG. 9a1 | FIG. 9b1 |
| H, L, W, | Height, length, width, mean | 245, 500, 160, | |
| HL | lamella height | 40 mm | |
| f | Centre excitation frequency | 120 kHz | |
| c_{S}, c_{A} | Sound speed in timber and air | 1000 m s⁻¹, | |
| | | 344 m s⁻¹ | |
| λ_{S}, λ_{A} | Wavelength in timber and air | 8.3, 2.9 mm | |
| r | Radius of active area of | 25 mm | |
| | transducers | | |
| T_{R} | Length of time window analyzed | 450 μs | |
| ΔH = HTR | Displacement in Y between | 40 mm | 80 mm |
| | IP and RP | | |
| θ_{I} | Angle of insonification | 4.8° | 8.8° |
| θ_{R} | Angle of refraction inside the | 14.0° | 26.6° |
| | sample | | |
| WTS, | Separation in X between | 185, 80 mm | |
| WRS | transducers and sample | | |
| HTS, HRS | Displacement in Y of ultrasound | 15, 7 mm | 29, 12 mm |
| | beam in air | | |

The transmitter transducer T was excited with a pulsed excitation consisting of a burst of five sinusoidal cycles at a fixed frequency of 120 kHz and 170 Vpp amplitude. The signals acquired by R were amplified by 40 dB with a low-noise, battery powered amplifier and band-pass filtered between 50 and 200 kHz with further 20 dB amplification. Then the time waveforms UW were digitized for each scan position with 2.5 MHz sampling rate and 14 bits digital resolution.

Specific noise reduction was implemented with a digital 80/160 kHz band-pass filter and a specific spatial averaging procedure, which associated to each waveform an average of the waveforms in a window of adjacent scanned positions (here 12×12 mm², corresponding to 36 pixels). Then, for each scanned pixel, the peak amplitude tracked continuously within a time window T_{R} of the recorded ultrasound waveforms UW was used as ultrasound wave signature of the presence or absence of glued timber defects: large amplitude values within T_{R} were associated to good ultrasound transmission P1 and small amplitude values to a blocking of the sound field P0, which in turn were associated to absence and presence of glued timber defects, respectively, in accordance with FIG. 3a.

The measurement results are represented as ultrasound images UI in normalized logarithmic scale which shows the internal state of the sample S. Light gray values correspond to large amplitude values, dark gray values to small amplitude values. Z and Y coordinates are directly associated with specific insonification positions in the lateral face FN1 along the length L and height H of the sample S, respectively. Dashed horizontal lines separate insonified lamellas in the ultrasound image UI with respect to the lateral face FN1. The pixel size/digital resolution of UI is equal to the scanning resolution, i.e. 1 mm in Y and 4 mm in Z.

The effective resolution of UI is of the order of magnitude of the diameter of the ultrasound transducers.

The amplitude measured in the ultrasound images UI at specific Y coordinates, such as profiles 9.1 to 9.10, is associated to specific propagation paths which interact with bonding planes B1, B2, . . . , Bi at specific width positions of S. For example, the amplitude at profile 9.6 describes the interaction of RF with the bonding plane B1 at half of the sample width W/2 (FIG. 9b1), whereas the amplitude at profile 9.7 describes the interaction of RF with the bonding plane B2 at half of the sample width W/2. Specific defect DP and defect-free positions GP are marked within the sample S corresponding to specific Z positions in UI.

A schematic representation of the expected ultrasound images SUI according to FIG. 3a, Eq2 and Eq 3 is provided for comparison purposes.

### Results and Discussion

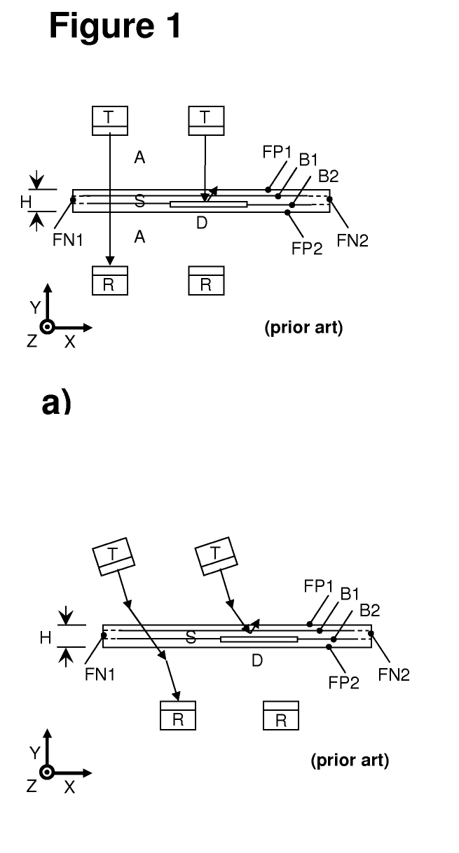

FIG. 9a2 shows results for an experiment (FIG. 9a1) in which ΔH was chosen equals to the height of a single timber lamella ΔH=HL. From Y positions 9.2 to 9.3, the refracted sound field RF is blocked by the lamination defect D in bonding plane B2 until half length L/2 of the sample S, which leads to a small ultrasound amplitude in the ultrasound image UI. For the other half length of the sample no defect is present in B2 so large ultrasound amplitude is observed in UI. From Y positions 9.1 to 9.2, large amplitude is measured at all Z positions, which indicates that no defects are present in bonding plane B1 and adjacent timber lamellas, with which RF interacts. The same between Y positions 9.3 and 9.4, therefore showing that no defect is present in B3 and adjacent timber lamellas. The same between Y positions 9.4 and 9.5 for B4. The measured ultrasound image UI (FIG. 9a2) therefore determines the extension of the defect D and associates it unequivocally to bonding plane B2, the setup moreover not being limited by the number of lamellas of the glulam beam and not requiring access to long side faces FL1 and FL2, as it would have been the case with the prior art described in FIG. 1. The measured UI (FIG. 9a2) image is in good agreement with the expected simulated SUI (FIG. 9a5) image.

Measured ultrasound waveforms UW are shown for defect DP (FIG. 9a3) and defect-free GP (FIG. 9a4) positions. As expected for negative detection, the wave propagation path P1 transmitted through the sample S is only present in GP. Large spurious signals PS, such as the wave path diffracted through air around the sample S are present for defect-free GP and defect DP positions, however, the use of a pulsed excitation signal allows filtering them out in time by evaluating the ultrasound amplitude only within the time window T_{R}.

FIG. 9b2 shows results for a second experiment (FIG. 9b1) in which ΔH was chosen equals twice the height of a single timber lamella ΔH=2 HL, and which allows an independent verification of the assessment provided by FIG. 9a2. From Y positions 9.6 to 9.8 the refracted sound field RF is blocked by the lamination defect D in bonding plane B2 until half length L/2 of the length, small ultrasound amplitude was accordingly observed in the ultrasound image UI. For the other half of the beam no defect is present, accordingly large amplitude values were observed in UI. From Y positions 9.8 to 9.9 large amplitude values were observed in UI, which indicates that no defect is present in bonding planes B3 and B4, across which the refracted sound field is transmitted. From Y positions 9.9 to 9.10 small amplitude values are obtained, since the refracted sound field RF is blocked by the long side face FL1 of the sample S for all Z positions. The measured ultrasound image UI (FIG. 9b2) therefore determines the extension of the defect D and associates it unequivocally to bonding plane B2, thus validating the results provided by FIG. 9a2. The measured UI (9b2) image is in good agreement with the expected simulated SUI (9b5) image. Measured ultrasound waveforms are shown at a defect position DP (FIG. 9b3) and a defect-free position GP (FIG. 9b4). Similarly to FIG. 9a3, the wave propagation path P1 transmitted through the sample S is only present in GP.

The natural variability of the sample S leads to amplitude uncertainties (typically <15 dB) at defect-free regions of UI. At defect regions of UI the amplitude reduction observed (typically >30 dB) is limited by spurious signals within T_{R} corresponding to propagation paths through the sample S without interaction with timber defects, for example, due e.g. to the finite diameter and side lobes of the ultrasound beam or due to channeling effects in the ring structure. In general, a compromise is required when choosing ΔH. Large ΔH values lead to longer propagation paths within the sample and thus to lower signal-to-noise ratio and higher amplitude uncertainty. Small ΔH values are generally subjected to larger spurious signals, due to the smaller Y-displacement HTR between insonification positions IP and re-radiation positions RP.

The use of ΔH=HL (FIG. 9a1) implies that a single defect D across the full width W of the sample S leads to reduced amplitude values in a one-lamella region of the ultrasound image UI (profiles 9.2 to 9.3 in FIG. 9a2), whereas for ΔH=2 HL (FIG. 9b1) a two-lamella region in the ultrasound image UI (profiles 9.6 to 9.8) shows small amplitude values for the same defect D. In general ΔH=K HL, with K typically an integer factor, associates to each bonding plane a K-lamella region in the ultrasound image UI. Large K increase the Y-extension of small-amplitude regions in the ultrasound images UI, which are then easier to distinguish from the natural variability of the sample. On the contrary, large K lead to overlaps between the assessment of K-adjacent bonding planes. For example, profile 9.6 in FIG. 9b2 contains simultaneously information about the interaction of the refracted sound field RF with B1 at middle width W/2 and with B2 at the right width edge.

For typical glued laminated timber geometries the assessment performs well for ΔH equals to the height of one to three timber lamellas.

In comparison with FIG. 9a2, FIG. 9b2 shows different amplitude variation patterns at defect-free regions, since ultrasound propagation occurs through different paths within the timber lamellas. However, the positions in the ultrasound images UI, where defect D blocks the refracted beam RF are consistent under consideration of the specific geometries for each experimental setup. The combination of several images obtained with different ΔH can therefore be used to verify and improve the robustness of the inspection according to the space diversity concept described above by means of FIG. 5.

### Example 2

### Positive Detection of Timber Defects

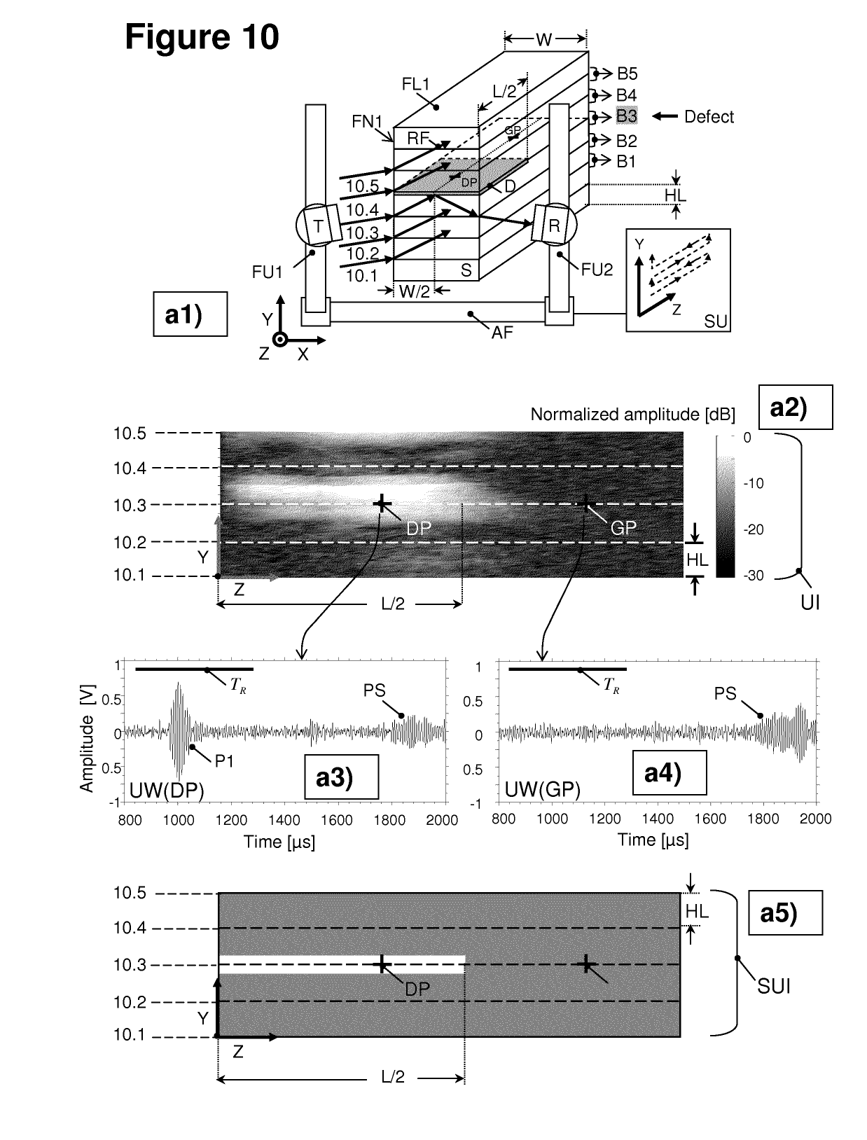

FIG. 10a1 shows an experimental implementation of the invention according to the positive detection ultrasound inspection setup described in FIG. 3b together with recorded ultrasound signals (FIG. 10a3, FIG. 10a4) for a typical glued laminated timber beam and imaging results for individual bonding planes (FIG. 10a2).

### Materials and Methods

The inspected sample S is a commercial glued laminated timber beam made from coniferous wood (Picea abies Karst), which dimensions are L=280, W=200, H=280. The mean height of the lamellas is HL=40 mm. A lamination defect D, specifically a saw cut, was introduced in the fourth lamination for half of the beam length (200×250 mm²).

The positive detection configuration of FIG. 10a1 was implemented with a similar experimental setup to FIG. 9a1. The transmitter T and receiver R transducers, the excitation signal, the settings of the acquisition equipment, the data evaluation and amplitude imaging procedure were the same as used in EXAMPLE 1. The transducers were attached to fixtures FU1 and FU2, as described in FIG. 7a, joined with an attachment frame AF, as described in FIG. 7b, and scanned as a single unit in Z and Y with a two-axis mechanical scanner SU in a raster fashion as described in FIG. 7c. The scanning steps were 1 mm steps in Y and 4 mm in Z. The transducers were configured according to the positive detection setup of FIG. 3b, with angle of incidence θ_{I} and relative positioning of both transducers fixed for all scanned positions. The geometric parameters of this setup were computed with Eq 2 and Eq 4 for inspection at middle sample width W/2 and are summarized in the following table:

| | | Values |
|---|---|---|
| Parameter | Description | FIG. 10 |
| H, L, W, | Height, length, width, mean lamella height | 280, 280, 200, |
| HL | | 40 mm |
| f | Centre excitation frequency | 120 kHz |
| c_{W}, c_{A} | Mean sound speed for timber and air | 1000 m s⁻¹, |
| | | 344 m s⁻¹ |
| λ_{W}, λₐ | Wavelength in timber and air | 8.3, 2.9 mm |
| r | Radius of active area of transducers | 25 mm |
| T_{R} | Length of time window analyzed | 450 μs |
| ΔH | Displacement in Y of RF within defect-free | 80 mm |
| | sample | |
| HTR | Displacement in Y between IP and RP | 0 mm |
| x | X-displacement between FN1 and DP | 100 mm |
| θ_{I} | Angle of insonification | 7.3° |
| θ_{R} | Angle of refraction inside the sample | 21.8° |
| WTS, | Separation in X between transducers and | 185, 80 mm |
| WRS | sample | |
| HTS, HRS | Displacement in Y of ultrasound beam in air | 24, 10 mm |

Large amplitude values were associated to good ultrasound transmission P1 and small amplitude values to a blocking of the sound field P0, which in turn were associated to the presence and absence of glued timber defects, respectively, in accordance with FIG. 3b. The results are presented as an ultrasound image UI and a schematic representation of simulated ultrasound transmission pattern SUI, in a similar way to FIG. 9.

### Results and Discussion

FIG. 10a2 shows experimental results for an experiment in which ΔH=80 mm was chosen as the width of two timber lamellas. At Y position 10.3, the ultrasound beam is reflected by the lamination defect D at middle width W/2 of bonding plane B3 and until half length L/2 of the sample, therefore, large amplitude values are observed in the ultrasound image UI. For the other half of the beam no timber defect is present and the ultrasound beam is transmitted through the sample, being re-radiated at a position where no receiver transducer is present, therefore a reduced amplitude (−25 dB) is observed in UI. This is as well the case for all Z at Y positions 10.1, 10.2, 10.4 and 10.5, which interact with bonding planes B-1, B-2, B-4 and B-5, respectively, where no defect is present. Specific time waveforms UW recorded for defect DP (FIG. 10a3) and defect-free GP (FIG. 10a4) positions are as well shown. The wave propagation path transmitted through the sample P1 is only present at defect positions (positive detection). Large spurious signals PS, such as the wave path diffracted through air around the sample are present for defect-free GP and defect DP positions, being filtered out in time by using pulsed excitation signals and evaluating ultrasound amplitude only within the time window T_{R}. The contrast between defect-free and defect positions is limited, similarly to negative detection, by wave propagation paths propagating through the timber lamellas without interaction with timber defect areas. A directive receiver transducer R allows filtering out wave propagation paths with different inclinations than the one reflected at the timber defect area, in order to achieve enough angular discrimination it is recommended that ΔH is equal or larger than the height of two timber lamellas.

As in EXAMPLE 1, the ultrasound image UI generated allows a clear identification of the glue line B3 along the height of the beam in which the defect is present and an assessment of the extension of the timber defect, the setup moreover not being limited by the number of lamellas of the glulam beam and not requiring access to long side faces FL1 and FL2, as it would have been the case with the prior art described in FIG. 1. The measured UI and simulated SUI images are in good agreement.

### Example 3

### Characterization of Total Re-Radiated Sound Field Scattered at Timber Defects

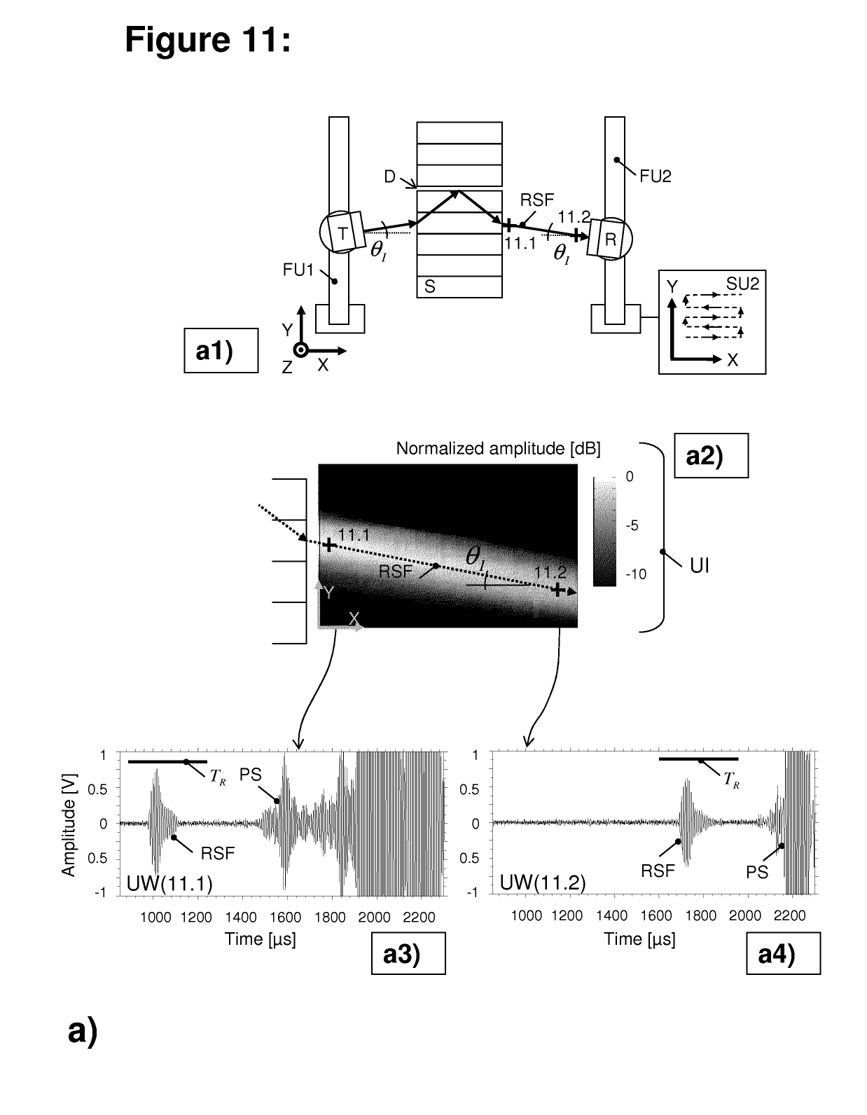

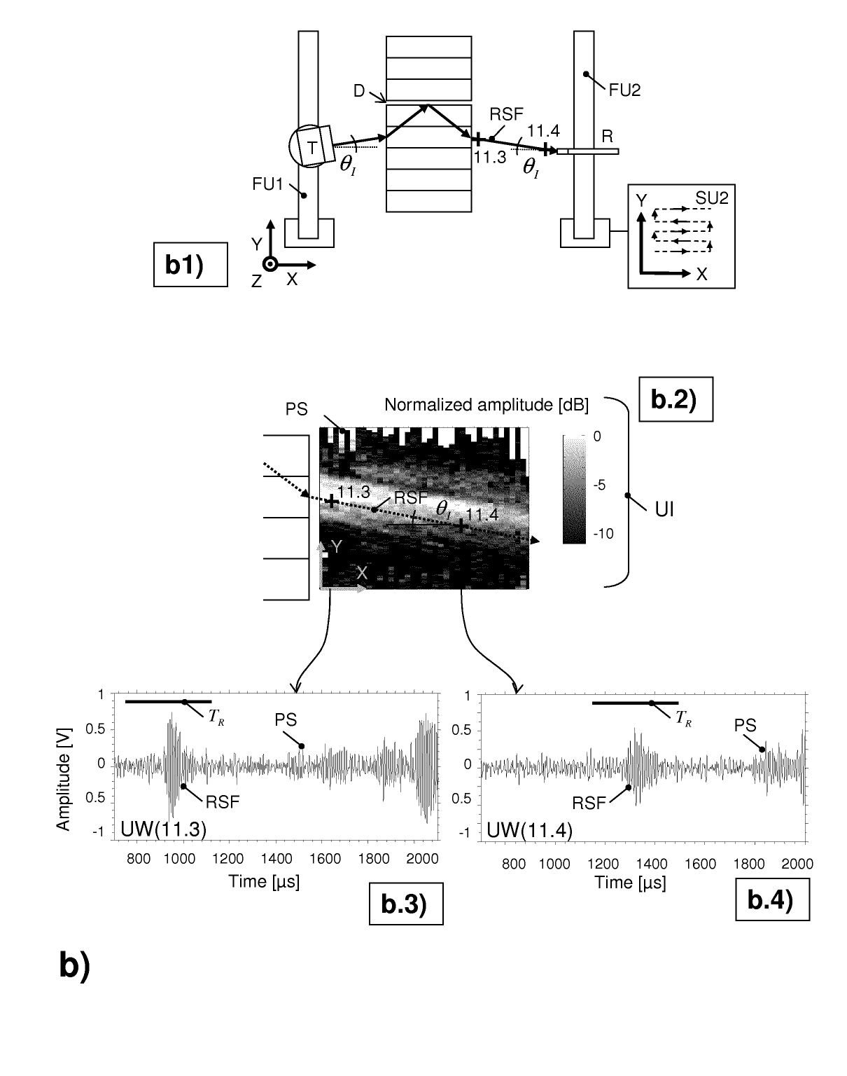

FIGS. 11a1 and 11b1 shows specific installations according to the sound field scanning setup described in FIG. 6c together with ultrasound images (FIGS. 11a2, 11b2) of the typical sound field distribution scattered at a timber defect area D and re-radiated into air, and ultrasound waveforms (FIGS. 11a3, 11a4, 11b3, 11b4) recorded at specific scan positions.

### Materials and Methods

The inspected sample S is a commercial glued laminated timber beam made from coniferous wood (Picea abies Karst.), which dimensions are L=320, W=135, H=300. The mean height of the lamellas is HL=35 mm. A lamination defect D, specifically an air gap, was introduced in the fourth lamination (135×320 mm²).

The sound field scanning configuration of FIG. 6b was implemented for a positive detection setup similar to FIG. 10. Two ultrasound transducers, namely transmitter T and receiver R were used. The transmitter T was a planar gas matrix piezoelectric composite (The Ultran Group Inc.) with r=25 mm, which was attached to a fixture FU1 as described in FIG. 7a, and was left fixed at middle beam length with position and orientation (98=10°) calculated with Eq 2 and Eq 4, in order to generate a reflection at middle width of the timber defect area D. The receiver R was mounted on a fixture FU2 as described in FIG. 7b, which was attached to a three-axis mechanical scanner SU2 and scanned in a raster fashion in the X and Y directions independently from T as described in FIG. 6c. For each scanned position ultrasound waveforms UW were recorded, and ultrasound images UI were generated by evaluating the peak amplitude within a time window T_{R}. The excitation signal, the settings of the acquisition equipment and the data evaluation and amplitude imaging procedure were the same used in EXAMPLE 1.

Two types of receiver transducer R were used. In a first implementation, which is shown in FIG. 11a1, a planar directive transducer of the same characteristics as T was used. The inclination of the receiver transducer was then adjusted to the same value θ_{I} as the transmitter transducer with mirroring in X, in accordance to the positive detection configuration of FIG. 3b. In a second implementation, which is shown in FIG. 11b1, a point omnidirectional receiver was implemented with a low-cost off-the-shelf microelectromechanic sensor (Knowles Electronics LLC) with a square active area of approx. 3×3 mm². The required electronics (polarization) were implemented within the casing of the transducer and battery powered in order to minimize electrical noise. No receiver inclination was required in this case, in accordance with the setup of FIG. 6c.

### Results and Discussion

FIG. 11a2 shows ultrasound amplitude images UI of a XY section of the ultrasound beam reflected at the timber defect area D and re-radiated into air RSF. FIG. 11a2 was measured with the directive receiver. No spatial averaging was used. As expected, the ultrasound beam emerges from the sample tilted with the same inclination as the incident ultrasound beam mirrored in X direction. The inclination of the re-radiated sound field RSF was measured on the ultrasound image as 10.2°, which is in good correspondence with the expected e, value. Specific time waveforms are shown at two positions 11.1 (FIG. 11a3) and 11.2 (FIG. 11a4). The time of arrival of the ultrasound signals increases with larger X coordinates due to an increase of the length of the wave propagation path in air between sample and receiver transducer. A pulsed excitation system and amplitude imaging with a moving time window allows separation of the ultrasound signals transmitted through the sample P1 from spurious signals PS, for example ultrasound waves diffracted through air around the sample. The directive transducer filters out spurious waves re-radiated off the transducer inclination.

FIG. 11b2 was measured with the point omnidirectional receiver. Spatial averaging with 5 pixels was used to reduce noise. The ultrasound image obtained UI (FIG. 11b2) is sharper than FIG. 11b2, due to the smaller active area of the receiver. Since ultrasound energy is acquired only in a small active area the signal level is generally lower than for the directive transducer, giving a lower signal-to-noise ratio than in FIG. 11a2, which is yet sufficient to accurately characterize the radiated sound field. In typical acquired time waveforms at positions 11.3 (FIG. 11b3) and 11.4 (FIG. 11b4) the wave paths propagated through the sample RSF can be clearly identified. The noisy pixels in UI correspond to spurious signals diffracted through air PS. There is room for further noise reduction by using e.g. pulse compression techniques.

These results demonstrate that full characterization of the time/space distribution of the sound field re-radiated through the sample is possible for an arbitrary beam excitation. Off-the-shelf omni-directional point sensors allow accurate characterization of the sound field with sufficient signal-to-noise ratio. Several such point sensors can be combined to implement multi-sensor receivers and electronic scanning according to the setup of FIG. 6d.

The sound field scattered by a defect a re-radiated in air RSF was successfully identified in the ultrasound images, in good agreement with the expected position and orientation according to Eq 1 to Eq 4, which validates the transducer positioning and orientation disclosed in FIG. 3 and FIG. 4. The measurement setups disclosed generate the desired wave propagation paths and interactions with defect areas and thus allow for non-destructive determination of defects in laminated structures.

### Example 4

### Numerical Simulation of Timber Defect Inspection

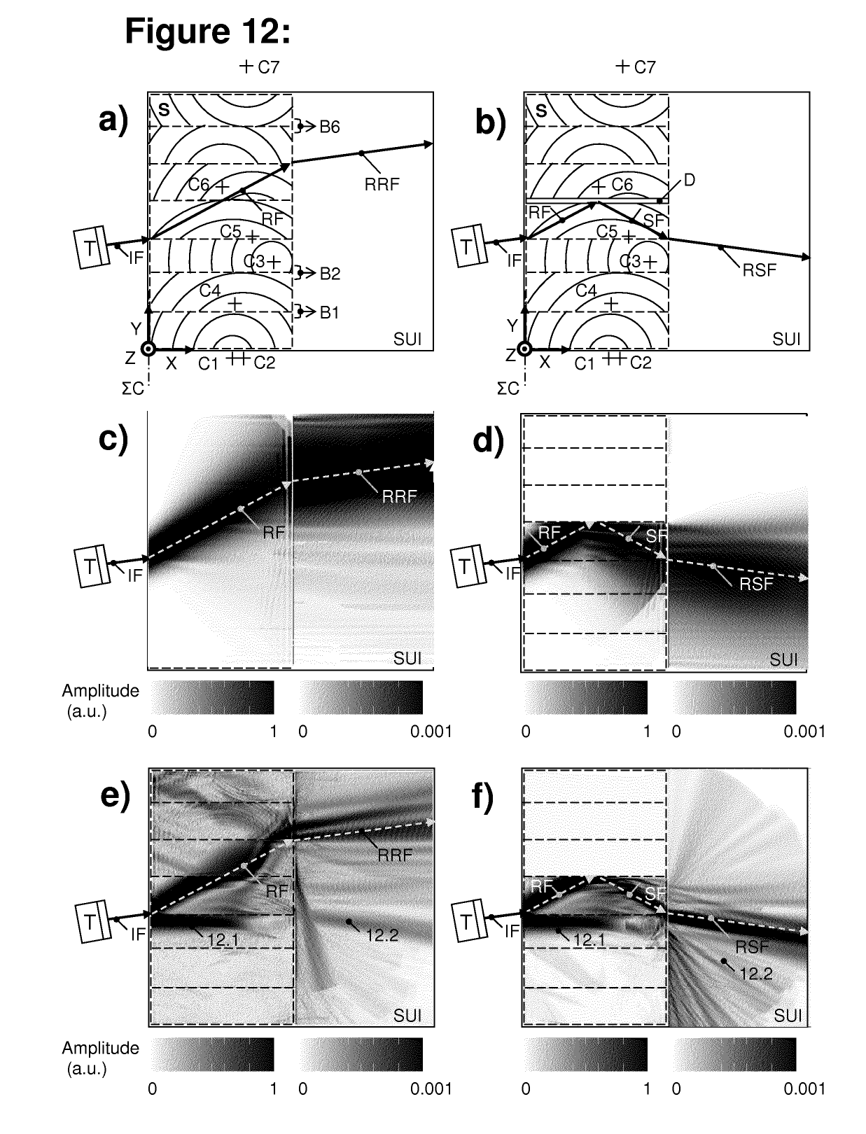

FIG. 12 shows a specific numerical implementation of the ultrasound wave propagation model described in FIG. 8a together with inner and re-radiated sound field distributions computed for a specific glued timber sample with and without lamination defects.

### Materials and Methods

The ultrasound wave coupling and propagation within a specific section of a glued timber sample S were calculated in a discretized domain of 912 cm² by using a two dimensional implementation of the Finite Difference Time Domain method (FDTD). FIG. 12a and FIG. 12b show a schematic representation of the computation domain. A specific rotated staggered grid well-known from cracked material simulation literature was used as direct discretization region (DDR) in order to discretize the discontinuities air-wood and the interior of sample S in a stable way. A voxel size of 200 μm×200 μm and a time discretization step of 100 ns were chosen to minimize numerical dispersion and ensure stability. A time window of 750 μs was analyzed. Only a thin air layer of 1 mm was discretized around the faces of the sample. Perfect matched layers of 10 voxels were implemented at the boundaries of the discretization domain in order to minimize numerical scattering. The incident sound field If in air in a surface ΣC in the vicinity of the sample was calculated for a single transmitter transducer T by using a point source projection PPΣTΣC based on elementary cylindrical sources. The inclination of the ultrasound beam IF generated by T was calculated according to Eq 2 for ΔH=80 mm, corresponding to the height of two timber lamellas HL. The total sound field re-radiated into air TRF (sum of RRF and RSF contributions) was calculated directly at specific positions of the discretization domain DDR, which is equivalent to using omnidirectional point receivers with radiating areas of the voxel size. Simulated ultrasound images SUI were generated by computing the peak amplitude value for each voxel of DDR.

The glued timber sample S modeled a commercial glued laminated timber beam, for which elastic properties were locally calculated by using a cylindrical orthotropic model. A nine component stiffness tensor referred to the principal axes of a wood stem (growth direction, perpendicular to year rings and tangential to year rings) was locally rotated according to the year ring curvature. The curvature centers C1, C2, . . . , C7 were determined individually for each lamella from optic scans of the end cross-section surfaces of the beam. In this particular example, damping and density variations were not considered. The bonding planes B1, B2, . . . B6 were 200 μm thick and were modeled with isotropic material by using the elastic properties of polyurethane. Numerical simulations were performed for a sample without timber defects (FIG. 12a,c,e) and for the same sample with a delamination defect D (200 μm thick air gap) in the bonding plane B4 (FIG. 12b, d, f). The results of the cylindrical orthotropic model (FIG. 12e, f) were compared with the ones obtained for an equivalent fluid model (FIG. 12c, d) which efficiently implements longitudinal wave coupling according to Snell's law. The wave propagation paths pre-calculated according to Eq 1 to Eq 4 are plotted with arrows (IF, RF, RRF, SF, RSF) for comparison.

### Results and Discussion

FIGS. 12c-f show simulated ultrasound images SUI of the stress field distribution within the sample S and re-radiated into air, represented in normalized amplitude units. The amplitude of the sound field re-radiated into air is, as expected, three-orders of magnitude smaller (0 . . . 0.001) with respect to the waves propagating through the glued timber sample (0 . . . 1), due to the inefficient coupling in interfaces air-wood. The wave propagation paths calculated with Eq 1 to Eq 4 are plotted with arrows (IF, RF, RRF, SF, RSF) for comparison. The calculated propagation paths accurately match to the ones simulated with Eq 1 to Eq 4. In the case of a sample without defects, which is shown in FIG. 12c, the ultrasound beam coupled into the sample IF is refracted at the interface ΣC, transmitted RF across the bonding plane B4 and re-radiated into air RRF. In the case of glued timber with defects, which is shown in FIG. 12d, the timber defect D blocks the transmission path RF and leads to a beam reflection SF which is re-radiated into air in a diverging direction RSF from RRF. By positioning a receiver transducer to capture RRF and RSF, negative (FIG. 3a) and positive (FIG. 3b) detection setups are simulated.

FIGS. 12e and 12f show results for the cylindrical orthotropic glued timber model. The wave propagation paths transmitted RRF and reflected RSF at glued timber defect areas are still present, their trajectories and beam spread being influenced by the ring structure. The calculated propagation paths are in good agreement with the ones simulated with Eq 1 to Eq 4. Additional wave propagation paths (e.g. 12.1) are coupled by mode conversion at the interfaces between air, timber lamellas and bonding planes together with guided modes in the year ring structure, which can lead to spurious re-radiated waves in air (e.g. 12.2). The wave propagation model enables selection of re-radiated wave paths associated to interactions with timber defects areas and is used to tune the experimental settings to account for deviations from the wave propagation paths precalculated with Eq 1 to Eq 4 due to the heterogeneity and anisotropy of wood structure.

These results demonstrate that computation and characterization of inner wave propagation paths within a glued timber sample S is possible for an arbitrary beam excitation. The direct propagation model DPM can be used to define the incident parameters of transmitter and receiver. The measurement setups disclosed in the invention generate the desired wave propagation paths and interactions with defect areas and thus allow for non-destructive determination of defects in laminated structures.

### Supplementary Technological Information on the Implementation of the Method Described by Invention

Several off-the-shelf transducer technologies efficiently couple or detect an ultrasound field in air at ultrasound center excitation frequencies f between 20 kHz and 5 MHz, for instance, specific piezoelectric composites, micromachined capacitive transducers, high frequency loudspeakers and microphones, microelectromechanic transducers, electrets and laser-doppler-vibrometers. The coupling is typically performed at ambient humidity, temperature and pressure. Modified gas coupling conditions, e.g. compressed or non-stationary gas, can also be applied for improving coupling characteristics.

The ultrasound excitation unit TU is fed with electrical signals, typically high-voltage pulses between 100 and 1000 Vpp are preferred in order to achieve sufficient ultrasound energy coupling and time filtering of undesired propagation paths, for instance, direct air transmission; continuous wave excitation is as well possible. The pulsed signals can be burst signals or chirp signals.

For certain transducer types, for instance, capacitive transducers, a continuous polarization voltage needs to be superimposed to the excitation signals. Additional noise reduction is achieved by means of a gated amplifier which is powered only in signal transmission periods. Electrical matching networks and output monitoring are implemented at electrical components and transducer interfaces in order to maximize output voltage or transference of power, minimize signal distortion and provide equalization feedback. The bandwidth of the electrical components is matched to the frequency range of the ultrasound transducers T, R.

A typical excitation waveform is a short (1-10 oscillations) pulsed burst at a fixed excitation frequency, optionally multiplied with a windowing function, for instance Hanning or Gaussian. Another common excitation is a pulsed signal with linearly increasing frequency within the bandwidth of the excited ultrasound transducers (chirp), which in combination with pulse compression techniques, reduces noise in received ultrasound signals.

Additional excitation coding strategies can be used, for instance, for wavelet decomposition or ultrasound signal equalization. In a multiple transducer system, specific signals are fed to each of the transducer elements; for example, variable apodization or delay laws are used to implement array phasing.

### Summary of the Invention According to FIG. 2

The invention describes a method and installation for non-destructive assessment of glued laminated structures S of large cross-section, such as glued laminated timber beams, which images the position and geometry of defects such as, in particular, lamination flaws (e.g. D1, D2), and allows for inspection of structural members of arbitrary height H and length L, and an individual assessment of specific bonding planes (e.g. B1, B2, B3), as well in situations with constrained access (e.g. 2.1) to the long side faces of the sample parallel to the bonding planes (e.g. FP1, FP2). The method is summarized by the following steps: a) Generation of an ultrasound beam IF in air A and coupling into the sample S through at least one lateral face FN1 of the sample which is essentially perpendicular to the inspected bonding planes, the ultrasound beam being tilted with a small inclination θ_{I} from the normal of the insonified surface so that an ultrasound beam RF is refracted into S with an inclination θ_{R}. b) Adjustment of the position and orientation of IF along predetermined paths in order to scan the entire glued laminated structural member. c) Acquisition of the sound field transmitted RF or reflected through the glued structural member and re-radiated into air (RSF, RRF). d) Extraction of wave signatures from the recorded ultrasound dataset describing the interaction of the ultrasound beam with defect areas, typically characterized by transmission of the ultrasound beam RF through defect-free regions and reflection or scattering SF at defect regions. e) Determination of individual maps UI for specific bonding planes (e.g. B1, B2, B3) and adjacent lamellas of mean height HL, in which defects (D1, D2) are imaged, based on predetermined relations between parameters of incident ultrasound beam, ultrasound wave signatures recorded for a specific portion ΔH of the height of the sample and type, position and geometry of defects. The installation for executing the method comprises: a) an air-coupled ultrasound beam generator consisting of a computerized arbitrary waveform generator (2.2), high power transmitter electronics (2.3) and an ultrasound excitation unit TU composed of a number of transducers (e.g. T1, T2) separated with a variable air gap WTS from surface FN1. b) A device, e.g. mechanical or electronic scanners (SU1, SU2) together with transducer positioning fixtures (FU1, FU2), which moves the ultrasound beam along defined trajectories and selects specific wave propagation paths by adjusting specific parameters, e.g. position, orientation and phasing, of transmitter and receiver ultrasonic transducers and excited and captured signals. c) An ultrasound sensor unit RU composed of a number of transducers (e.g. R1, R2, R3) separated with variable air gaps WSR from surface FN2, low-noise electrical signal amplification and filtering electronics (2.4) and an analog to digital converter (2.5) for time/space digitization of a dataset of ultrasound waveforms UW for each scanned pixel. d) Specific signal processing algorithms implemented on a microprocessor (2.6) for noise and spurious signal reduction and ultrasound wave signature extraction. e) Specific direct DPM and inverse IPM ultrasound wave propagation models for optimization of transducer positions and orientations, correlation of acquired wave signatures with ultrasound propagation paths within glued laminated sample, and inversion of sound field at defect areas, from which type, position and geometry of defects are derived. The method and installation are validated with modeling and experimental results.

1. Air coupled ultrasonic contactless method for non-destructive determination of defects in a laminated structure (S) with a width (W) and a multiplicity of n lamellas with intermediate n−1 bonding planes (Bi), wherein at least one transmitter (T) in a fixed transmitter distance (WTS) radiates ultrasound incident sound fields (IF) at multiple positions and at least one receiver (R) in a sensor distance (WSR) is receiving re-radiated ultrasound fields (RRF, RSF) at multiple positions relative to the laminated structure (S), comprising the steps of:
coupling the incident sound field (IF) of an ultrasound beam from an active area of the at least one transmitter (T) through a lateral face (FN1) after passing a gas medium (A) in a fixed transmitter distance (WTS) into the laminated structure (S),
whereas the normal of the lateral face (FN1) is at least essentially perpendicular to the normals of the bonding planes (Bi),
whereas the ultrasound incident sound field (IF) is tilted with an inclination angle θ_{I} from 0° to 20° from the normal of the lateral face (FN1),
receiving re-radiated refracted and/or scattered sound fields (RRF, RSF) with the at least one receiver (R) in the sensor distance (WSR) after propagation of the ultrasound beam through the width (W) of the laminated structure (S) within a reduced portion of the total height (H) and length (L) of the laminated structure (S) after passing a re-radiated lateral face (FN2) and a gas medium (A), before:
ultrasound wave signatures are extracted by subsequent low-noise electrical signal amplification, filtering, analogue to digital conversion and signal processing of ultrasound datasets, before:
generation and representation of ultrasound images (UI) of the gluing state of each bonding plane is performed, such that defects are determined independently from the total height (H) and length (L) of the laminated structure (S) and defects are determined without access to the long side faces (FP1, FP2, . . . ) of the laminated structure (S).

2. Air coupled ultrasonic contactless method according to claim 1, wherein the lateral face (FN1) and the re-radiated lateral face (FN2) are two different opposing faces of the laminated structure (S).

3. Air coupled ultrasonic contactless method according to claim 1, wherein the incident sound field (IF) comprising a center excitation frequency (f) is chosen to achieve a smaller wavelength in the laminated structure (S) than a mean lamella height (HL).

4. Air coupled ultrasonic contactless method according to claim 3, wherein the fixed transmitter distance (WTS) satisfies WTS>0.5r² c_{A}⁻¹ f, where f is the center excitation frequency of the ultrasound incident sound field (IF), c_{A} is the sound velocity in air and r is the radius of the active area of the transducer (T).

5. Air coupled ultrasonic contactless method according to claim 1, wherein the inclination angle θ_{I} is calculated by
$${θ}_{I} = arcsin ( \frac{{c}_{A}}{{c}_{S}} sin ( arctan \frac{Δ H}{W} ) )$$
where (W) is the width of the laminated structure (S), (C_{A}) is the sound velocity in air, (c_{S}) is the sound velocity in the material of the laminated structure (S), and (ΔH) is a parameter varying between zero and the width (W) before the transducer (T, R) are inclined accordingly.

6. Air coupled ultrasonic contactless method according to claim 1, wherein the separation between the intersection of the normal of the at least one transmitter (T) with the lateral face (FN1) and the Intersection of the normal of the at least one receiver (R) with the re-radiated lateral face (FN2) is estimated by
$$HTR = Δ H ( 1 - \frac{2 x}{W} )$$
where (W) is the width of the laminated structure (S), (ΔH) is a parameter varying between zero and the width (W), and (x) is another parameter varying between zero and the width (W).

7. Air-coupled ultrasonic contactless method according to claim 5, wherein ΔH is a multiple of lamella height (HL).

8. Air coupled ultrasonic contactless method according to claim 1, wherein the incident sound field (IF) is a pulse with center frequency (f).

9. Air coupled ultrasonic contactless method according to claim 8, wherein the sensor distance (WSR) between the receiver transducer (R) and re-radiated lateral face (FN2) is adjusted before the coupling of the incident sound field (IF) to the length of the analyzed time window (T_{R}) matching WSR>0.5c_{A}T_{R}, where (c_{A}) is the sound velocity in air.

10. Air coupled ultrasonic contactless method according to claim 1, wherein measured ultrasound datasets are represented as a multiplicity of time vs. amplitude waveforms, and ultrasound images (UI) are generated by determining for each waveform amplitude and phase within analyzed time window (T_{R}).

11. Air coupled ultrasonic contactless method according to claim 1, wherein a multiplicity of transmitter (T) and/or a multiplicity of receiver (R) forming arrays are used.

12. Air coupled ultrasonic contactless method according to claim 1, wherein the at least one transmitter (T) is scanned at least parallel to the lateral face (FN1) and/or the at least one receiver (R) is scanned at least parallel to the re-radiated lateral face (FN2) with a scanning system unit (SU) as a fix unit.

13. Air coupled ultrasonic contactless method according to claim 10, wherein the scanning system unit (SU) is implemented either mechanically, with multiple axes scanner or robotic arms or is implemented electronically, with array phasing technology.

14. Air coupled ultrasonic contactless method according to claim 10, wherein the at least one transmitter (T) and the at least one receiver (R) are scanned synchronized to each other in Y and Z directions.

15. Air coupled ultrasonic contactless method according to claim 1, wherein the laminated structure (S) is moved relative to the at least one transmitter (T) and/or the at least one receiver (R).

16. Air coupled ultrasonic contactless method according to claim 1, wherein the at least one transmitter (T) and the at least one receiver (R) are adjusted relative to each other in such a way, that the normals of active areas of transducer (T, R) faces are parallel to each other.

17. Air coupled ultrasonic contactless method according to claim 1, wherein the at least one transmitter (T) and the at least one receiver (R) are adjusted relative to each other in such a way, that the normals of active areas of transducer (T, R) faces are angled including an angle of (180°−2*inclination angle θ_{I}).

18. Air coupled ultrasonic contactless method according to claim 1, wherein the at least one transmitter (T) and the at least one receiver (R) are adjusted relative to each other in such a way, that the normals of active areas of transducer (T, R) faces are angled including an angle of 2*inclination angle θ_{I}.

19. Air coupled ultrasonic contactless method according to claim 1, wherein transmitter (T) and/or receiver (R) with active areas possessing radii (r) of the active areas in the order of half of the mean lamella height (HL) are chosen.

20. Air coupled ultrasonic contactless method according to claim 1, wherein directive, omnidirectional, focused or planar transducers (T, R) are chosen.

21. Air coupled ultrasonic contactless method according to claim 20, wherein single or multi element ultrasound transducers (T, R) are used.

22. Air coupled ultrasonic contactless method according to claim 1, wherein measurement parameters as transmitter distance (WTS), sensor distance (WSR), separation between the intersection of the normal of the transmitter and the lateral face (FN1) and the intersection of the normal of the receiver (R) with the re-radiated lateral face (FN2), inclination angle θ_{I} and excitation frequency f are estimated by simulation and computation of a direct ultrasound wave propagation model (DPM) on the basis of a defect free laminated structure, before insonification of the lateral face (FN1) with an incident sound field (IF).

23. Air coupled ultrasonic contactless method according to claim 1, wherein after a measurement, measured ultrasound images (UI) or ultrasound datasets are interpreted by inverting the data with an inverse ultrasound wave propagation model (IPM) and associating the results with defect positions, types and geometries within the laminated structure (S).

24. Air coupled ultrasonic contactless method according to claim 22, wherein the direct ultrasound wave propagation model (DPM) incorporates:
a) synthesis of ultrasound beam in the vicinity of coupling lateral faces (FN1, FN2) by decomposition of transducer near field in elementary point excitations,
b) computation of coupling and wave propagation phenomena within laminated structure (S) in a defined discretization domain,
c) projection of the re-radiated sound field to specific receiver (R) positions,
d) incorporation of transfer functions associated to the at least one transducer (T) and/or electric equipment, and
e) selection of wave paths with specific orientation and polarization.

25. Air coupled ultrasonic contactless method according to claim 23, wherein the inverse ultrasound wave propagation model (IPM) incorporates:
a) measurement and/or simulation of re-radiated sound fields for calibration sample (S_{C}) in at least a receiver plane (ΣR) parallel to the re-radiated lateral face FN2,
b) measurement of re-radiated sound fields for an equivalent sample (S) with undetermined defects,
c) calculation of difference re-radiated sound field by comparison of re-radiated sound fields measured obtained in a) and b),
d) mirroring with respect to time axis and signal processing of the difference re-radiated sound field,
e) computation of coupling through lateral face FN2 and wave propagation of processed difference re-radiated sound field within calibration laminated structure (S_{C}) in a defined discretization domain, and
f) interpretation of sound field computed within the discretization domain by extraction of wave signatures as wave convergences with subsequent signal processing and associating the results with defect positions, types and geometries within the laminated structure (S).

26. Air coupled ultrasonic contactless method according to claim 22, wherein for repeated measurements of the same laminated structure (S) the optimized parameters determined by measurement and successive simulation, are later used for further measurements.

27. Air coupled ultrasonic contactless method according to claim 1, wherein after repeated measurements of the same laminated structure (S), for which the same or different measurement parameters are used, measured ultrasound images (UI) or ultrasound datasets are interpreted by identifying differences and/or similarities in repeated measurements and associating the results with defect positions, types and geometries within the laminated structure (S).

28. Air coupled ultrasonic contactless method according to claim 1, wherein the laminated structures (S) are comprising wood and/or wood composites, and the laminated structures (S) are inspected in a production line or in situ at a construction site.

29. An installation for performing an air coupled ultrasonic contactless method for non-destructive determination of defects in a laminated structure (S) having a width (W) between opposing lateral faces (FN1, FN2) and having a multiplicity of n lamellas with intermediate n−1 bonding planes (Bi), the installation comprising:
at least one transmitter (T) disposed relative to the laminated structure (S) at a fixed transmitter distance (WTS) for radiating ultrasound incident sound fields (IF) at multiple positions for rendering the lateral faces (FN1, FN2) insonified,
at least one receiver (R) disposed at a sensor distance (WSR) for receiving re-radiated ultrasound fields (RRF, RSF) at multiple positions relative to the laminated structure (S),
a transducer fixture attached to the at least one transmitter (T) and/or the at least one receiver (R), the transducer fixture comprising a targeting device, positioning means and pivoting means, wherein the targeting device, which includes a first line laser (7.10) and a second line laser (7.11), which are disposed for generation of a reference point-in a contactless way in the form of a light cross (7.14) on the insonified lateral faces (FN1, FN2) of the laminated structure (S) for simplified transducer positioning with respect to the lateral faces (FN1, FN2) and bonding planes (Bi).

30. (canceled)
